(19) United States
(12) Patent Application Publication
(10) Pub. No.: US 2024/0189199 A1
(43) Pub. Date: Jun. 13, 2024

(54) WHITENING COMPOSITION USEFUL FOR THE PREPARATION OF COSMETIC FORMULATIONS

(71) Applicant: SENSIENT COSMETIC TECHNOLOGIES, SAINT-OUEN L'AUMONE (FR)

(72) Inventors: Paola Andrea SANCHEZ CELY, Valle del Cauca CALI (CO); Tara MARTIN, TAVERNY (FR); Patricia VESQUE, FRANCONVILLE (FR); Pamela EKLOU, COLOMBES (FR); Sophie HONG, CERGY (FR)

(21) Appl. No.: 18/554,422

(22) PCT Filed: Apr. 4, 2022
(86) PCT No.: PCT/EP2022/058843

§ 371 Date: Oct. 6, 2023

(30) Foreign Application Priority Data
Apr. 9, 2021 (FR) 2103646

(51) Int. Cl. A61K 8/27; A61K 8/19; A61K 8/26; A61Q 1/04

(52) U.S. Cl. CPC A61K 8/27; A61K 8/19; A61K 8/26; A61Q 1/04; A61K 2800/30; A61K 2800/434

(57) ABSTRACT

Disclosed is a whitening composition including a mixture of zinc oxide and of boron nitride and, if appropriate, a whitening agent selected from kaolin optionally calcined, cerium oxide, a citrate salt, mica and a mixture thereof. The whitening composition is useful for imparting opacifying, whitening and/or covering properties to a cosmetic formulation, as a replacement for titanium dioxide.

### CROSS-REFERENCE TO RELATED APPLICATIONS

This application is the US national stage of PCT/EP2022/058843, filed Apr. 4, 2022 and designating the United States, which claims the priority of FR 2103646, filed Apr. 9, 2021. The entire contents of each foregoing application are incorporated herein by reference.

### BACKGROUND OF THE INVENTION

### Field of the Invention

The present invention relates to a whitening composition useful for the preparation of cosmetic formulations having a whitening, covering and/or opacifying effect.

### Description of the Related Art

Titanium dioxide (TiO₂) is used in many applications ranging from paints, coatings, textiles to cosmetics and pharmaceuticals. Titanium dioxide is one of the pigments with the highest refractive index, and the whitening, opacifying, covering and tint-lightening properties thereof are unmatched at the moment. However, the use of titanium dioxide is now being questioned because of:
being banned for food-processing by the French government,
the upcoming classification thereof according to the CLP (Classification, Labelling, Packaging) Regulation ((EC) Regulation No 1272/2008 of the European Parliament on the classification, labelling and packaging of chemicals and mixtures) as a category 2 carcinogenic substance by inhalation, for mixtures marketed in powder form containing 1% or more of particles with an aerodynamic diameter of ≤10 μm,
the impact thereof in case of oral exposure.

There is thus a need to develop a replacement product having whitening, opacifying and/or covering properties accordingly consistent with the needs of different cosmetic applications.

Furthermore, the preparation method for cosmetic formulations comprises mixing the different constituents of the cosmetic formulation. Such method is facilitated when “all-in-one” products comprising a plurality of the constituents of the cosmetic formulation can be used instead of the constituents taken alone. Thereby, the method comprises fewer steps, involves fewer starting materials, and the losses of sometimes costly constituents, which may occur during the separate introduction of each constituent can be minimized.

There is thus a further need to provide an “all-in-one” product for the preparation of cosmetic formulations having a whitening, covering and/or opacifying effect.

### SUMMARY OF THE INVENTION

To this end, the invention relates to a whitening composition comprising (or even consisting of):
from 0 to 30% by weight, preferentially from 1 to 30% by weight, of a whitening agent selected from kaolin optionally calcined, cerium oxide, a citrate salt, mica and a mixture thereof,
from 70 to 100% by weight, preferentially from 70 to 99% by weight, of a mixture of zinc oxide and of boron nitride,
the proportions being with respect to the cumulative weight of whitening agent, of zinc oxide and of boron nitride,
wherein the ratio between the weight of zinc oxide and the weight of boron nitride is from 0.9 to 5.0, preferentially from 0.9 to 4.6, preferentially from 0.9 to 3.0, preferentially from 0.9 to 2.9, preferentially from 0.9 to 2.5, more preferentially from 0.9 to 2.0, the whitening composition being free of titanium dioxide.

In the present application, the concentrations of constituents are expressed by weight.

The whitening composition comprises 75 to 100% by weight of a mixture of zinc oxide and of boron nitride with respect to the combined weight of whitening agent, of zinc oxide and of boron nitride.

The mixture of zinc oxide and of boron nitride imparts whitening effects and advantageously makes it possible to replace titanium dioxide. In order to avoid the aforementioned undesirable effects of titanium dioxide, the whitening compositions according to the invention, and preferentially the cosmetic formulations which comprise them, are free of titanium dioxide.

A cosmetic formulation comprising both boron nitride and zinc oxide is more opaque than a cosmetic formulation comprising only boron nitride, or than a cosmetic formulation comprising only zinc oxide, which shows a synergistic effect of the combined use of the boron nitride and of the zinc oxide on the imparted opacity.

Boron nitride has a very high tint-lightening and whitening power. However, when used alone, boron nitride imparts a dull tint to cosmetic formulations, less saturated for the foundation application (parameter C*<20) and yellowish for the lipstick application (parameter b*>2) compared to the use of titanium dioxide for which the tint remains intense. The combined use of zinc oxide and boron nitride makes it possible to compensate and reduce such phenomenon, or even to prevent same in the cosmetic formulation into which the whitening composition is introduced.

Boron nitride generally neutralizes the blue hue imparted by zinc oxide to the cosmetic formulation into which same is introduced, and zinc oxide neutralizes the yellow hue imparted by boron nitride to the cosmetic formulation into which same is introduced, thus making it possible to obtain a more neutral tint for the cosmetic formulation comprising both zinc oxide and boron nitride, which is particularly advantageous for certain makeup applications. In addition, zinc oxide neutralizes the low saturation value imparted by boron nitride to the formulation into which same is introduced, which is also particularly advantageous for certain makeup applications.

Preferentially, the boron nitride is in the form of particles with hexagonal configuration. Such configuration makes it possible indeed to obtain a good whitening and opacifying power. The morphology of the particles is preferentially quasi-graphitic or turbostratic, particularly preferentially turbostratic, such morphologies imparting the best opacifying power.

Preferentially, the boron nitride is in the form of particles with a mean diameter, as measured by laser diffraction, of 1 to 30 μm, preferentially of 2 to 8 μm. The smaller the mean diameter, the more opacifying properties boron nitride has.

The ratio between the weight of zinc oxide and the weight of boron nitride is from 0.9 to 5,0, preferentially from 0.9 to 4.6, preferentially from 0.9 to 3.0, preferentially from 0.9 to 2.9, preferentially from 0.9 to 2.5, more preferentially from 0.9 to 2.0, which optimizes the opacity imparted to the cosmetic formulation by the whitening composition.

Preferentially, the zinc oxide is in the form of particles with a mean diameter, as measured by laser diffraction, of 30 nm to 20 μm, preferentially of 100 nm to 3 μm.

The cosmetic formulation may include or be free of whitening agents other than zinc oxide and boron nitride. When present, such agents are preferentially chosen amongst kaolin optionally calcined, cerium oxide, citrate salt, mica and a mixture thereof, present in a concentration of 0 to 30% by weight with respect to the cumulative weight of whitening agent, zinc oxide and boron nitride. When the whitening composition comprises a mixture of two or more of the whitening agents, the concentration of 0 to 30% by weight corresponds to the cumulative concentration thereof.

The citrate salt is preferentially calcium citrate or sodium citrate, particularly preferentially tricalcium citrate.

### Preferentially:

the kaolin optionally calcinedis in the form of particles with a mean diameter, as measured by laser diffraction, of 0.4 to 15 μm, preferentially of 0.4 to 2.0 μm, and/or
citrate salt is in the form of particles with a mean diameter, as measured by laser diffraction of 1.0 to 10 μm, preferentially of 1.0 to 4.0 μm, and/or
mica is in the form of particles with a mean diameter, as measured by laser diffraction, of 1.0 to 10 microns, preferentially of 2.0 to 7.0 μm, and/or
cerium oxide is in the form of particles with a mean diameter, as measured by laser diffraction of 1.0 to 50 μm, preferentially of 15 to 30 μm.

Preferentially, in addition to being free of titanium dioxide, the whitening composition is free of one, two, or three of the whitening agents selected from bismuth oxychloride, magnesium oxide, and calcium carbonate. Indeed, the inventors observed that the whitening agents degrade the properties of the whitening composition into which same are introduced.

When present, the particles of boron nitride and/or zinc oxide and/or whitening agent(s) may have undergone a hydrophobic, hydrophilic, lipophobic, lipophilic and/or anti-photocatalytic surface treatment.

Hydrophobic treatments are carried out e.g. with:
a silicone derivative preferentially chosen from methicone, dimethicone, hydrogen dimethicone, triethoxypolydimethylsiloxyethyl hexyl dimethicone, polypropylsilsesquioxane
a dimethicone derivative preferentially chosen from cetyl dimethicone, methicone/dimethicone copolymer, dimethiconol stearate, cetyl dimethicone,
a silane derivative preferentially selected from triethoxycaprylylsilane, N-octadecyl-triethoxysilane, 2-[methoxy(polyethyleneoxy)6-9 propyl]trimethoxysilane, poly(diethylsiloxane), methoxytrimethylsilane, dimethoxydimethylsilane, hydrogen methyl polysiloxane, dimethylpolysiloxane,
an acrylate derivative, preferentially acrylate dimethicone copolyol,
an olefin derivative preferentially polyethylene and oxidized polyethylene,
a ceramide, preferentially hydroxypropyl bispalmitamide MEA,
a fluorinated derivative preferentially selected from ammonium C6-16 perfluoroalkylethyl phosphate, C9-13 perfluoroalcohol phosphoric acid, sodium perfluorohexylethyl phosphate, perfluoroalkyl ethyl triethoxysilane, DEA C8-C18 perfluoroalkylethyl phosphate,
a phospholipid derivative preferentially chosen from lecithin and hydrogenated lecithin,
a wax preferentially chosen from beeswax, carnauba wax, candelilla wax, rose wax and other floral waxes such as mimosa, lavender, chamomile.
a fatty acid derivative preferentially selected from jojoba oil, hydrogenated stearyl olive ester, avocado butter, glycolipids (pseudozyma tsukubaensis/olive oil/glycerin/soya protein ferment), magnesium myristate, magnesium stearate, aluminum dimyristate, aluminum stearate, cetyl alcohol, isostearyl sebacate, isopropyl titanium triisostearate, caprylic/capric triglyceride, hydrogenated camellia oil, hydrogenated vegetable oil, aloe vera oil extract, coconut oil.
an amino acid derivative preferentially chosen from lauroyl lysine, stearoil glutamic acid, sodium myristoyl glutamate, disodium stearoyl glutamate, aluminum lauroyl glutamate,
an acid preferentially chosen from phytic acid, hyaluronic acid and stearic acid.

Hydrophilic treatments are carried out e.g. with:
a dimethicone derivative preferentially chosen from PEG-12 dimethicone, PEG-8 methyl ether triethoxysilane,
a silane derivative, preferentially methoxy PEG-10 propyltrimethoxysilane,
a fatty acid derivative preferentially sodium glycerophosphate,
an olefin, preferentially sodium C14-16 olefin sulfonate,
a polysaccharide preferentially chosen from microcrystalline cellulose, chitosan, algin, galactoarabinan,
silica.

The lipophilic treatment is carried out, e.g., with an acrylate copolymer.

The anti-photocatalytic treatment is carried out e.g. with aluminum and/or silica.

The embodiments described hereinabove apply to each of the alternatives described hereinafter.

The whitening composition according to the invention is particularly suitable for being used in a cosmetic formulation useful as a compact powder, lipstick, soap and/or foundation, preferentially useful as a compact powder, lipstick and/or foundation.

According to a first alternative, the whitening composition comprises (or even consists of):
from 0 to 10% by weight, preferentially from 0 to 5% by weight, in particular from 1 to 5% by weight, of a whitening agent chosen from kaolin optionally calcined, cerium oxide, and a mixture thereof
from 90 to 100% by weight, preferentially from 95 to 100% by weight, in particular 95 to 99% by weight, of a mixture of zinc oxide and boron nitride, the proportions being with respect to the cumulative weight of whitening agent, of zinc oxide and of boron nitride, 30
wherein the ratio between the weight of zinc oxide and the weight of boron nitride is from 0.9 to 3.0, preferentially from 0.9 to 2.9, preferentially from 0.9 to 2.5, preferentially from 1.3 to 3.0. preferentially from 1.5 to 3.0.

A whitening composition according to the first alternative is particularly suitable for use in a cosmetic formulation which is useful as a compact powder, lipstick and/or foundation, in particular as a foundation.

A proportion of 90 to 100% by weight, preferentially 95 to 100% by weight, of the mixture of zinc oxide and boron nitride, with respect to the combined weight of whitening agent, zinc oxide and boron nitride, allows the whitening composition to impart improved opacifying, covering and lightness properties to the cosmetic formulation into which same is introduced.

In a first embodiment of the first alternative, the whitening composition comprises (or even consists of):
from 30 to 40% by weight, preferentially 36% by weight, of boron nitride, and
from 60 to 70% by weight, preferentially 64% by weight, of zinc oxide, the proportions being with respect to the cumulative weight of zinc oxide and of boron nitride. Preferentially, in the first embodiment of the first alternative, the whitening composition is free of kaolin optionally calcined, cerium oxide, citrate salt and mica.

In a second embodiment of the first alternative, the whitening composition comprises (or even consists of):
from 1 to 10% by weight, preferentially from 3 to 7% by weight, of a whitening agent selected from kaolin optionally calcined, cerium oxide and a mixture thereof,
from 20 to 40% by weight, preferentially from 24% to 38% by weight, of boron nitride, and
from 55 to 75% by weight, preferentially from 57% to 71% by weight, of zinc oxide, the proportions being with respect to the cumulative weight of whitening agent, of zinc oxide and of boron nitride,
where the ratio between the weight of zinc oxide and the weight of boron nitride is from 0.9 to 3.0. preferentially from 1.3 to 3.0.

Preferentially, the whitening composition consists of:
5% by weight of a whitening agent chosen from kaolin optionally calcined, cerium oxide and a mixture thereof, preferentially cerium oxide,
26% by weight of boron nitride, and
69% by weight of zinc oxide.

Preferentially, the whitening composition consists of:
kaolin optionally calcined, cerium oxide and a mixture thereof, 5% by weight of a whitening agent chosen from kaolin optionally calcined,
36% by weight of boron nitride, and
59% by weight of zinc oxide.

According to a second alternative, the whitening composition comprises (or even consists of):
from 0 to 25% by weight, preferentially from 1 to 25% by weight, of a whitening agent selected from kaolin optionally calcined, a citrate salt and a mixture thereof, and
from 75 to 100% by weight, preferentially from 75 to 99% by weight, of a mixture of zinc oxide and of boron nitride,
the proportions being with respect to the cumulative weight of whitening agent, of zinc oxide and of boron nitride,
where the ratio of the weight of zinc oxide to the weight of boron nitride is 1.5 to 3.0.

A whitening composition according to the second alternative is particularly suitable for being used in a cosmetic formulation which is useful as a compact powder, lipstick and/or foundation, in particular as a lipstick. Cosmetic formulations useful as foundations generally require more coverage and tint lightening than cosmetic formulations useful as lipsticks. The percentage of boron nitride for a whitening composition intended for a cosmetic formulation useful as lipsticks can thus be reduced compared with same of the first alternative.

In a first embodiment of the second alternative, the whitening composition comprises (or even consists of):
from 25 to 30% by weight, preferentially 27% by weight, of boron nitride, and
from 70 to 75% by weight, preferentially 73% by weight, of zinc oxide, the proportions being with respect to the cumulative weight of zinc oxide and of boron nitride. Preferentially, in the first embodiment of the second alternative, the whitening composition is free of kaolin optionally calcined, cerium oxide, citrate salt and mica.

In a second embodiment of the second alternative, the whitening composition comprises (or even consists of):
from 10 to 25% by weight, preferentially from 15 to 25% by weight, of a whitening agent selected from kaolin optionally calcined, a citrate salt and a mixture thereof, and
from 20 to 40% by weight, preferentially from 25% to 30% by weight, of boron nitride, and
from 50 to 60% by weight, preferentially from 50% to 55% by weight, of zinc oxide, the proportions being with respect to the cumulative weight of whitening agent, of zinc oxide and of boron nitride, where the ratio of the weight of zinc oxide to the weight of boron nitride is 1.3 to 3.0.

Preferentially, the whitening composition consists of:
20% by weight of a whitening agent chosen from kaolin optionally calcined, a citrate salt and a mixture thereof,
28% by weight of boron nitride, and
52% by weight of zinc oxide.

Preferentially, the whitening composition comprises (or even consists of):
20% by weight of a whitening agent chosen from kaolin optionally calcined, a citrate salt and a mixture thereof,
26% by weight of boron nitride, and
54% by weight of zinc oxide,
the proportions being with respect to the cumulative weight of whitening agent, of zinc oxide and of boron nitride,

According to a third alternative, the whitening composition comprises (or even consists of):
from 0 to 10% by weight, preferentially from 1 to 10% by weight, of a whitening agent selected from kaolin optionally calcined, a citrate salt, mica and a mixture thereof,
from 90 to 100% by weight, preferentially from 90 to 99% by weight, of a mixture of zinc oxide and of boron nitride,
the proportions being with respect to the cumulative weight of whitening agent, of zinc oxide and of boron nitride,
where the ratio between the weight of zinc oxide and the weight of boron nitride is from 3.5 to 5.0. preferentially from 4.0 to 5.0.

A whitening composition according to the third alternative is particularly suitable for use in a cosmetic formulation which is useful as a compact powder, lipstick, soap and/or foundation, in particular as a soap, in particular as a solid soap.

The bulk whiteness imparted is more significant for the solid soap application than the lightening of a tint related to the pigment load thereof or the coverage on skin or lips. For the above reason, a high percentage of zinc oxide is used in the boron nitride/zinc oxide mixture (high ratio between the weight of zinc oxide and the weight of boron nitride).

Preferentially, the whitening composition comprises (or even consists of):
from 0 to 10% by weight, preferentially from 2 to 8% by weight, of a whitening agent selected from kaolin optionally calcined, a citrate salt, mica and a mixture thereof,
from 10 to 30% by weight, preferentially from 13% to 22% by weight, of boron nitride, and
from 70 to 90% by weight, preferentially from 70% to 85% by weight, of zinc oxide, the proportions being with respect to the cumulative weight of whitening agent, of zinc oxide and of boron nitride,
where the ratio between the weight of zinc oxide and the weight of boron nitride is from 3.5 to 5.0. preferentially from 4.0 to 5.0.

In a first embodiment of the third alternative, the whitening composition comprises (or even consists of):
5% by weight of a whitening agent chosen from kaolin optionally calcined, a citrate salt, mica and a mixture thereof,
18 to 19% by weight of boron nitride, and
76 to 77% by weight of zinc oxide, the proportions being with respect to the cumulative weight of whitening agent, of zinc oxide and of boron nitride,

In a second embodiment of the third alternative, the whitening composition comprises (or even consists of):
15 to 20% by weight of boron nitride, and
80 to 85% by weight of zinc oxide, the proportions being with respect to the cumulative weight of zinc oxide and of boron nitride. Preferentially, in the second embodiment of the third alternative, the whitening composition is free of kaolin optionally calcined, cerium oxide, citrate salt and mica.

According to a fourth alternative, the whitening composition comprises (or even consists of):
from 0 to 30% by weight, in particular from 1 to 30% by weight, preferentially from 1 to 10% by weight, of a whitening agent selected from kaolin optionally calcined, cerium oxide and a mixture thereof,
from 70 to 100% by weight, in particular from 70 to 99% by weight, preferentially from 90 to 99% by weight, of zinc oxide and boron nitride, the proportions being with respect to the cumulative weight of whitening agent, of zinc oxide and of boron nitride,
wherein the ratio between the weight of zinc oxide and the weight of boron nitride is from 0.9 to 3.0, preferentially from 0.9 to 2.9, preferentially from 0.9 to 2.5, preferentially from 0.9 to 2.0.

A whitening composition according to the fourth alternative is particularly suitable for being used in a cosmetic formulation which is useful as a compact powder, lipstick and/or foundation, in particular as a compact powder.

In a first embodiment of the fourth alternative, the whitening composition comprises (or even consists of):
from 33 to 40% by weight, preferentially 36% by weight, of boron nitride, and
from 60 to 67% by weight, preferentially 64% by weight, of zinc oxide, the proportions being with respect to the cumulative weight of zinc oxide and of boron nitride. Preferentially, in the first embodiment of the fourth alternative, the whitening composition is free of kaolin optionally calcined, cerium oxide, citrate salt and mica.

In a second embodiment of the fourth alternative, the whitening composition comprises (or even consists of):
from 0 to 10% by weight, preferentially 5% by weight, of a whitening agent chosen from kaolin optionally calcined, cerium oxide, and a mixture thereof, preferentially kaolin,
from 32 to 40% by weight, preferentially 36% by weight, of boron nitride, and
from 55 to 63% by weight, preferentially 59% by weight, of zinc oxide, the proportions being with respect to the cumulative weight of whitening agent, of zinc oxide and of boron nitride,

In a third embodiment of the fourth alternative, the whitening composition comprises (or even consists of):
from 15 to 30% by weight, preferentially 30% by weight, of a whitening agent chosen from kaolin optionally calcined, cerium oxide, and a mixture thereof, preferentially kaolin,
from 30 to 37% by weight, preferentially 36% by weight, of boron nitride, and
from 33 to 40% by weight, preferentially 34% by weight, of zinc oxide, the proportions being with respect to the cumulative weight of whitening agent, of zinc oxide and of boron nitride,

The whitening composition is generally prepared by mixing the constituents thereof, e.g. in a horizontal Lodige mixer with shares and lump breakers.

The whitening composition according to the invention is suitable for being introduced into a cosmetic formulation.

The present invention thus further relates to the use of a whitening composition according to the invention, for the preparation of a cosmetic formulation. The invention further relates to a process for preparing a cosmetic formulation comprising adding a whitening composition according to the invention to a cosmetic formulation.

The whitening composition according to the invention corresponds to an “all-in-one product” suitable for being introduced into a cosmetic formulation. The use of the whitening composition is economically advantageous since it is possible to introduce the different components thereof in only one step, which leads to a saving of time and can minimize the losses of compounds during production.

The inventors have discovered that a cosmetic formulation comprising the whitening composition according to the invention has a whitening, opacifying and/or covering effect, preferentially whitening, opacifying and covering effects at the same time.

According to a second subject matter, the invention relates to the use of a whitening composition, as defined hereinabove, for imparting opacifying, whitening and/or covering properties, preferentially opacifying, whitening and covering properties, to a cosmetic formulation.

The invention further relates to a process for improving the opacifying, whitening and/or covering properties of a cosmetic formulation comprising the addition thereto of a whitening composition according to the invention.

The CIE Lab L*a*b* method can be used for performing colorimetric measurements. The L* parameter indicating lightness and the a* and b* parameters indicating tint along two axes: the green-red axis for a* and the blue-yellow axis for b*. Negative values in the b* axis indicate an increasingly blue color and positive values an increasingly yellow color. Similarly, negative values in the a* axis indicate an increasingly green color and positive values an increasingly red color. The dE (delta E, ΔE* or ΔEab) measures the color difference between a color 1 and a color 2 according to the following formula:

ΔE*=\sqrt{(ΔL*)²+(Δa*)²+(Δb*)²}
wherein:
ΔL* is the difference in the L* parameter between color 1 and color 2,
ΔA* is the difference in the a* parameter between color 1 and color 2, and
Δb* is the difference in the b* parameter between color 1 and color 2.

The covering property of the whitening composition is the ability thereof to cover another color on the skin or on the lips. It is determined by applying a cosmetic formulation comprising the whitening composition to the skin, typically on the forearm on the inner part, or on the lips and classifying same as “covering”, “moderately covering”, “slightly covering” or “not covering”.

The opacifying property of the whitening composition is the ability thereof to intercept light. The opacifying properties of a cosmetic formulation comprising the whitening composition can be determined by applying same to a black and white contrast card (e.g. a Black & White Leneta card) with an automatic film-stretcher preferentially set to a thickness of 156 microns, then measuring the opacity of the film on the black surface of the contrast card and the opacity of the film on the white surface of the contrast card. The opacity can be measured by reflectance with a spectrophotometer (e.g. with a Konica Minolta CM-5 spectrophotometer).

The measure of opacity in % is given by the following formula

$$\frac{Measurement of opacity on a black surface}{Measurement of opacity on a white surface} \times 100$$

The method is similar to the method described in ISO 2471 of 2008 for the determination of opacity on paper background for the paper and board industry.

The opacity measurement of less than 50% corresponds to a transparent film and the opacity measurement of more than 50% corresponds to an opaque film. For an application of the cosmetic formulation as a foundation, an opacity measurement greater than 88% is required.

Furthermore, a cosmetic formulation comprising the whitening composition according to the invention is likely to make the tint paler. It concerns the ability of the whitening composition to whiten, to lighten the hue, to make paler the tint of a hue. Such ability is evaluated by means of the parameter L* defined hereinabove.

According to a third subject matter, the invention relates to a cosmetic formulation comprising the whitening composition defined hereinabove.

The cosmetic formulation can have any form: solid, possibly powdery, liquid, gel. It may concern an aqueous, oily or emulsion formulation (oil-in-water or water-in-oil emulsion e.g.). The cosmetic formulation can be a cleaning product (e.g. soap or shampoo) or a make-up product (in particular a powder, preferentially a compact powder, a foundation or a lipstick).

According to a first alternative, the cosmetic formulation is a foundation and comprises the whitening composition according to the first alternative defined hereinabove. The cosmetic formulation comprises in particular from 11 to 30% by weight, preferentially from 13 to 20% by weight, of the whitening composition according to the first alternative defined hereinabove with respect to the weight of the cosmetic formulation.

According to a second alternative, the cosmetic formulation is a lipstick and comprises the whitening composition according to the second alternative defined hereinabove. The cosmetic formulation comprises in particular from 10 to 30% by weight, preferentially from 12 to 20% by weight, of the whitening composition according to the second alternative defined hereinabove with respect to the weight of the cosmetic formulation.

According to a third alternative, the cosmetic formulation is a soap, preferentially a solid soap, and comprises the whitening composition according to the third alternative defined hereinabove. The cosmetic formulation comprises in particular from 0.1 to 2.0% by weight of the whitening composition according to the third alternative defined hereinabove with respect to the weight of the cosmetic formulation.

According to a fourth alternative, the cosmetic formulation is a compact powder and comprises the whitening composition according to the fourth alternative defined hereinabove. The cosmetic formulation comprises in particular from 10 to 30% by weight of the whitening composition according to the fourth alternative defined hereinabove. The invention is illustrated by the following examples.

### DETAILED DESCRIPTION

### Examples

In the examples, the following raw materials were used:
Boron Nitride (brand name CARESS BN02) from Bent Tree Industries.
Anatase Titanium dioxide (brand name Unipure White LC981) from Sensient Cosmetic Technologies
Zinc oxide from Sensient Cosmetic Technologies
Bismuth oxychloride (brand name Bismuth oxychloride 3N BI-OCLI-03) from American Elements
Cerium oxide (brand name Cerium Oxide CE-0X-02R-P) by American Elements Kaolin (brand name Imercare opaque) by Imerys
Magnesium oxide (brand name Magnesium Oxide heavy) from Paul Lohmann Tricalcium citrate (brand name Tricalcium citrate M1098) from Jungbunzlauer
MICA (brand name Mica 8 R2041) from Sensient Cosmetic Technologies
Mixture of hydroxyapatite and calcium carbonate (brand name Omyaskin 100-OG) from Omya

In the examples which follow, the expressions “formula”, “formulation” and “cosmetic formulation” are equivalent.

The different formulas were evaluated in terms of opacity, whitening power-lightness (L*), coverage, tint (a*, b*) and saturation (C*) according to the parameters most relevant to each application and detailed hereinafter.

To take the measurements L, a, b of the different formulas, a Konica Minolta CM-5 spectrophotometer with CM-S100W Spectra Magic NX software was used. In order to measure the different colorimetric parameters, the foundation and lipstick formulas were poured onto a transparent plastic cup.

The opacifying properties were determined by the method described hereinabove. Using a film-stretcher, if appropriate a heating film-stretcher for lipstick formulas, a 156-micron thick film was stretched over a black and white contrast card (a Black & White Leneta contrast card). A Konica Minolta CM-5 spectrophotometer with CM-S100W Spectra Magic NX software was used for acquiring the opacity measurements of the film.

In order to determine the covering properties of the formulas, eight panelists tested the different formulas on the front of the forearm on the inner part and classified the properties as “covering”, “moderately covering”, “slightly covering” or “not covering”.

Negative bounds are formulas free of any whitening compound. As detailed hereinbelow, in all cases, the negative bounds give much lower coverage, opacity and lightness results than the formulas according to the invention comprising a whitening composition.

### Example 1: Whitening Compositions for Foundation

### 1.1. Preparation of Whitening Compositions

The whitening compositions having the composition detailed in Table 1 were prepared.

TABLE 1
Compositions of the whitening compositions, the
proportions being expressed by weight/weight

| | | BLEND | BLEND | BLEND | BLEND | BLEND | BLEND | BLEND |
|---|---|---|---|---|---|---|---|---|
| | BLEND | 7 FDT | 8 | 8 NEW | 9 | 9 NEW | 10 | 10 NEW |
| | 4 FDT | (comparative) | FDT | FDT | FDT | FDT | FDT | FDT |
| Boron | 36 | 12 | 15 | 26 | 36 | 36 | 31 | 35 |
| nitride | | | | | | | | |
| Zinc oxide | 64 | 83 | 75 | 69 | 44 | 59 | 49 | 45 |
| Bismuth | — | 5 | — | — | — | — | — | — |
| oxychloride | | | | | | | | |
| Cerium | — | — | 10 | 5 | — | — | — | — |
| oxide | | | | | | | | |
| Kaolin | — | — | — | — | 20 | 5 | — | — |
| Magnesium | — | — | — | — | — | — | 20 | — |
| oxide | | | | | | | | |
| Tricalcium | — | — | — | — | — | — | — | 20 |
| citrate | | | | | | | | |
| ZnO/BN | 1.78 | 6.92 | 5.00 | 2.65 | 1.22 | 1.64 | 1.58 | 1.29 |
| ratio | | | | | | | | |

### Procedure for Making Each BLEND:

Mixing using a commercial Waring spice mill from Cuisinart PRO-PREP CHOPPER GRINDER CHOP tank with a stirring of 2×15 s (scraping between 2 turns). 1.2. Preparation of the water-in-oil TW1920 Total adequacy cosmetic formulations with a silicone environment and useful as foundation

### 1.2.1. Choice of the Most Suitable Whitening Compositions

The water-in-oil cosmetic formulations with a silicone environment and useful as a foundation having the composition detailed in Table 2 were prepared.

TABLE 2
Compositions of the of the water-in-oil TW1920 Total adequacy cosmetic formulations with a silicone
environment and useful as a foundation, the proportions being expressed by weight/weight

| | | | | | | | Formulation | |
|---|---|---|---|---|---|---|---|---|
| | | | | | | | with 10% | |
| | | | | | (comparative) | | TiO₂ | FDT |
| | | | | | Formulation | Formulation | (positive | formulation |
| | | | | FDT formulation | with 10% | with 10% ZnO | bound or | (negative |
| Phase | Brand name | INCI | Supplier | according to the invention | boron nitride | (comparative) | standard) | bound) |
| A | Purolan IHD | Isohexadecane | Lanxess | 2.60 | 2.60 | 2.60 | 2.60 | 2.24 |
| | Crodamol | Triethylhexanoin | Croda | 7.50 | 7.50 | 7.50 | 7.50 | 6.48 |
| | GTEH | | | | | | | |
| | Ceraphyl | Isostearyl | Ashland | 0.50 | 0.50 | 0.50 | 0.50 | 0.44 |
| | 375 | neopentanoate | | | | | | |
| | KF 6017 | PEG-10 | Shin Etsu | 4.50 | 4.50 | 4.50 | 4.50 | 3.89 |
| | | Dimethicone | | | | | | |
| | Silamer HC | Cetyl PEG/PPG- | Sensient | 5.20 | 5.20 | 5.20 | 5.20 | 4.5 |
| | | 10/1 Dimethicone | | | | | | |
| | | (and) | | | | | | |
| | | Triethylhexanoin | | | | | | |
| | | (and) Hexyl laurate | | | | | | |
| | | (and) Polyglyceryl- | | | | | | |
| | | 4-isostearate | | | | | | |
| B | Bentone Gel | Isododecane (and) | Elementis | 14.80 | 14.80 | 14.80 | 14.80 | 12.8 |
| | ISD V. | Disteardimonium | | | | | | |
| | | Isododecane (and) | | | | | | |
| | | Hectorite (and) | | | | | | |
| | | Hectorite | | | | | | |
| C | Pure water | Aqua | VWR | 30.65 | 30.65 | 30.65 | 30.65 | 31.66 |
| | Glycerin | Glycerin | AMI | 8.00 | 8.00 | 8.00 | 8.00 | 6.92 |
| | Salt | Sodium chloride | | 1.00 | 1.00 | 1.00 | 1.00 | 0.87 |
| | Euxyl PE | Phenoxyethanol | Schulke | 0.30 | 0.30 | 0.30 | 0.30 | 0.27 |
| | 9010 | (and) | | | | | | |
| | | Ethylhexylglycerin | | | | | | |
| D | Covabead | Methylmethacrylate | Sensient | 1.00 | 1.00 | 1.00 | 1.00 | 0.86 |
| | LH 170 | crosspolymer | | | | | | |
| E | Dow | Cyclopentasiloxane | Dow Corning | 12.00 | 12.00 | 12.00 | 12.00 | 2.8 |
| | Corning 245 | | | | | | | |
| | fluid | | | | | | | |
| | Unipure | CI 77492 | Sensient | 1.25 | 1.25 | 1.25 | 1.25 | 1.39 |
| | Yellow | | | | | | | |
| | LC182 | | | | | | | |
| | Unipure | CI 77491 | Sensient | 0.45 | 0.45 | 0.45 | 0.45 | 0.5 |
| | Red LC381 | | | | | | | |
| | Unipure | CI 77499 | Sensient | 0.25 | 0.25 | 0.25 | 0.25 | 0.28 |
| | Black | | | | | | | |
| | LC989 | | | | | | | |
| | One of the | CI 77891 (Titanium | Sensient | 10.00 | 0 | 0 | 0 | 0 |
| | BLENDS | dioxide), Boron | | | | | | |
| | from Table 1 | Nitride, Zinc Oxide, | | | | | | |
| | | any additional | | | | | | |
| | | component | | | | | | |
| | | specified in Table 1 | | | | | | |
| | Caress | Boron nitride | Bent Tree | 0 | 10.00 | 0 | 0 | 0 |
| | BN02 | | Industries | | | | | |
| | Zinc oxide | Zinc oxide | | 0 | 0 | 10.00 | 0 | 0 |
| | Unipure | Titanium dioxide | Sensient | 0 | 0 | 0 | 10.00 | 0 |
| | White | | Cosmetic | | | | | |
| | LC981 | | Technologies | | | | | |
| F | Dow | Cyclopentasiloxane | Dow Corning | 0 | 0 | 0 | 0 | 24.1 |
| | Corning 245 | | | | | | | |
| | fluid | | | | | | | |

### Procedure:

Homogenize Phase A with the stirrer for 5 min at 500 rpm. Disperse phase B into Phase A, while stirring for 10 minutes at 800 rpm. Add phase C to A+B, with gentle stirring at the beginning and then increase stirring to 800 rpm for 10 min.

Add phase D to phase A+B+C, while stirring.

Grind, using a tricylinder (Montceram Exakt 50i triclylinder), the pigments of phase E with phase F if [there is a] phase F, then add phase E+F to the rest of the formula at 500 rpm.

The formulation was produced either with 10% TiO₂ (positive bound or standard), or with 10% of each BLEND (formulation according to the invention), or with 10% boron nitride (comparative), or with 10% zinc oxide (comparative), or in the absence of whitening compound (negative bound).

For the standard, Unipure White LC981 titanium dioxide was chosen because same is a reference for anatase titanium dioxide, the latter being more covering than rutile titanium dioxide.

The opacity and lightness parameters, the tint parameter (which comprises the parameters a and b* and the saturation which will include the parameter C*) of the cosmetic formulation are the most determining parameters for the foundation application.

### 1.2.1.1. Viscosity

Table 3 below gives the viscosities measured on the cosmetic formulations of Table 2.

TABLE 3
Viscosities (cP) measured with a Brookfield viscosimeter
DV2T LVTJO Needle LV3 viscosity TW 1920 with 10% white

| | Formula including: | | | | | | | | |
|---|---|---|---|---|---|---|---|---|---|
| | BLEND | BLEND | BLEND | BLEND | BLEND | BLEND | | | |
| Speed | 4 | 7 FDT | 8 | 9 | 10 | 11 | ZnO | BN | LC981 |
| (rpm) | FDT | (comparative) | FDT | FDT | FDT | FDT | (comparative) | (comparative) | (Standard) |
| 30 | 2700 | 1752 | 3492 | 1716 | 2576 | 2696 | 3492 | 1620 | 1652 |
| 20 | 3264 | 2130 | 4368 | 2124 | 3288 | 3180 | 4326 | 1914 | 2010 |
| 10 | 4512 | 2880 | 6300 | 2892 | 5076 | 4308 | 6228 | 2520 | 2868 |
| 5 | 6960 | 4008 | 8472 | 3528 | 7992 | 6480 | 9768 | 3264 | 4704 |

The formula containing zinc oxide has a higher viscosity than the boron nitride formula, but regardless of the ingredients used in the formula, the viscosity is within the usual range for a foundation.

### 1.2.1.2. Opacity and Coverage

Table 4 below provides the opacity results of the cosmetic formulations listed in Table 2.

TABLE 4
Opacity of cosmetic formulations

| | | | Opacity | |
|---|---|---|---|---|
| | | | (ISO | |
| | | | 2471) | Coverage |
| Formula | TiO₂ LC981 (standard) | White | 100.46 | covering |
| comprising: | in the absence of | White | 87.94 | not covering |
| | whitening compound | | | |
| | (negative bound) | | | |
| | ZnO (comparative) | White | 98.6 | covering |
| | BN (comparative) | White | 97.75 | covering |
| | BLEND 4 FDT | White | 98.82 | covering |
| | BLEND 7 FDT | White | 86.3 | not covering |
| | (comparative) | | | |
| | BLEND 8 FDT | White | 97.32 | covering |
| | BLEND 9 FDT | White | 98.12 | covering |
| | BLEND 10 FDT | White | 96.52 | moderately |
| | | | | covering |
| | BLEND 11 FDT | White | 83.32 | not covering |
| | BLEND 8 NEW FDT | White | 97.73 | covering |
| | BLEND 9 NEW FDT | White | 100.15 | covering |

The cosmetic formulation comprising BLEND 4 FDT (consisting of boron nitride and of zinc oxide) gives better opacity results than the formulations comprising the raw materials individually (cosmetic formulation wherein the whitening compound is either boron nitride or zinc oxide), which shows a synergistic effect of boron nitride and of zinc oxide.

BLEND 10 FDT and BLEND 11 FDT, the concentrations of a mixture of zinc oxide and of boron nitride of which are only 80%, impart less opacity and coverage to the formulation into which same are introduced. Increasing the concentration of the mixture of zinc oxide and of boron nitride within the whitening composition improves the opacity imparted, as shown by comparing the results obtained with BLEND 8 NEW and 9 NEW (ZnO+BN concentration of 95%) with the results of BLEND 8 and 9, respectively (ZnO+BN concentration of 80%).

Among the formulations tested comprising multi-component whitening compositions, the cosmetic formulations comprising BLEND 4 FDT, BLEND 8 NEW FDT, BLEND 9 NEW FDT and BLEND 9 FDT are the formulations leading to the best opacity. BLEND 9 NEW FDT imparts the formulation with an opacity almost at the level of the opacity imparted by titanium dioxide.

### 1.2.1.2. LAB Color Measurements on Cup

Table 5 below gives the colorimetric measurements measured on the cosmetic formulations listed in Table 2.

TABLE 5
Colorimetric measurements (average over three measurements) of the different formulas in a L*a*b*CIE 1976 color space.

| | | | | | | | dE/ | dE/ |
|---|---|---|---|---|---|---|---|---|
| | | L*(D65) | a*(D65) | b*(D65) | C*(D65) | h(D65) | Bound+ | Bound− |
| Cosmetic | LC981 (Standard) | 54.69 | 13.54 | 20.33 | 24.42 | 56.33 | / | / |
| formulation | in the absence of whitening compound (negative | 33.90 | 18.98 | 29.11 | 34.76 | 56.89 | 23.21 | / |
| comprising | bound) | | | | | | | |
| | BLEND 4 | 50.50 | 15.21 | 20.89 | 25.84 | 53.94 | 4.55 | 18.90 |
| | FDT | | | | | | | |
| | BLEND FDT 7 (comparative) | 47.83 | 15.99 | 22.40 | 27.53 | 54.47 | 7.58 | 15.74 |
| | BLEND 8 | 48.87 | 15.75 | 21.48 | 26.63 | 53.76 | 6.33 | 17.11 |
| | FDT | | | | | | | |
| | BLEND 8 NEW | 52.65 | 13.78 | 17.79 | 22.50 | 52.24 | 3.26 | 22.51 |
| | FDT | | | | | | | |
| | BLEND 9 FDT | 48.57 | 15.76 | 22.25 | 27.26 | 54.69 | 6.78 | 16.51 |
| | BLEND 9 NEW FDT | 54.23 | 14.95 | 19.95 | 24.93 | 53.15 | 1.53 | 22.66 |
| | BLEND 10 FDT | 45.96 | 15.15 | 15.90 | 21.96 | 46.38 | 9.92 | 18.29 |
| | BLEND 11 FDT | 48.64 | 13.64 | 14.67 | 20.03 | 47.08 | 8.28 | 21.32 |
| | BN (comparative) | 56.80 | 12.06 | 14.23 | 18.66 | 49.73 | 6.62 | 28.17 |
| | ZnO (comparative) | 47.82 | 15.88 | 21.47 | 26.70 | 53.51 | 7.34 | 16.18 |

The composition comprising only boron nitride (formulation BN) imparts a better lightness to the formulation into which it is introduced than titanium dioxide (which is also observable with the naked eye). However, boron nitride lacks saturation (parameter C*<20), imparting a dull tint to foundation formulations, which is not suitable for the use of the formulation in a foundation.

Zinc oxide used alone (ZnO formulation) does not impart any significant effect on lightness.

BLEND 4 FDT is the formulation with the best results. Zinc oxide removes the “dull” side brought in by boron nitride.

The lightness and coverage results of BLEND 7 FDT (comparative—ZnO/BN ratio of 6.9) are not satisfactory. BLEND 10 FDT and 11 FDT (concentrations of zinc oxide and of boron nitride mixture of 80% only) are not [satisfactory] either.

BLENDS FDT 4, 8 and 9 impart the formulation into which same are introduced with the best results in terms of lightness, which is confirmed by observations with the naked eye. The lightness is not as good as the lightness imparted by titanium dioxide (standard), but in comparison with the results of the formula without titanium dioxide (negative bound), the whitening compositions used at 10% by weight with respect to the weight of the formulation confer a lightness which is close to 80% of the results obtained with titanium dioxide (standard).

Increasing the concentration of the mixture of zinc oxide and of boron nitride within the whitening composition improves the lightness imparted, as shown by the comparison of the results obtained with BLEND 8 NEW and 9 NEW (ZnO+BN in a concentration of 95%) with the results of BLEND 8 and 9, respectively (ZnO+BN in a concentration of 80%).

Given the opacity and colorimetric results, BLENDS 4, 8 NEW and 9 NEW have the best performance in terms of opacity, coverage and whitening power.

1.2.2. Determination of the Optimum Concentration of Whitening Composition within the Cosmetic Formulation

In order to find the optimal percentage for use, formulas with BLEND 4 to 12.5, 15 and 20% were prepared. The standard formulation used for the comparison always remains the formulation described hereinabove (formulation with 10% TiO2 (positive bound or standard) in Table 2) with 10% titanium dioxide, which corresponds to the average percentage of use in the marketed formulas.

The cosmetic formulations with compositions shown in detail in Table 6 were prepared.

TABLE 6
Compositions of the of the water-in-oil TW1920 Total adequacy cosmetic formulations
with a silicone environment and useful as a foundation, the proportions being expressed
by weight/weight, with a variable concentration of whitening composition BLEND 4

| | | | | 12.5% | 15% | 20% |
|---|---|---|---|---|---|---|
| Phase | Brand name | INCI | Supplier | formulation | formulation | formulation |
| A | Purolan IHD | Isohexadecan | Lanxess | 2.02 | 2.02 | 1.70 |
| | Crodamol GTEH | Triethylhexanoin | Croda | 5.83 | 5.83 | 4.92 |
| | Ceraphyl 375 | Isostearyl neopentanoate | Ashland | 0.4 | 0.4 | 0.32 |
| | KF 6017 | PEG-10 Dimethicone | Shin Etsu | 3.50 | 3.50 | 3.0 |
| | Silamer HC | Cetyl PEG/PPG-10/1 Dimethicone | Sensient | 4.05 | 4.05 | 3.41 |
| | | (and) Triethylhexanoin | | | | |
| | | (and) Hexyl laurate (and) | | | | |
| | | Polyglyceryl-4-isostearate | | | | |
| B | Bentone Gel ISD V. | Isododecane (and) | Elementis | 11.52 | 11.52 | 9.71 |
| | | Disteardimonium Hectorite | | | | |
| | | (and) Hectorite | | | | |
| C | Pure water | Aqua | VWR | 28.50 | 28.50 | 24.04 |
| | Glycerin | Glycerin | AMI | 6.23 | 6.23 | 5.25 |
| | Salt | Sodium chloride | Oxoid | 0.78 | 0.78 | 0.65 |
| | Euxyl PE 9010 | Phenoxyethanol | Schulke | 0.24 | 0.24 | 0.2 |
| | | (and) Ethylhexylglycerin | | | | |
| D | Covabead LH 170 | Methylmethacrylate crosspolymer | Sensient | 0.78 | 0.78 | 0.65 |
| E | Dow Corning 245 fluid | Cyclopentasiloxane | Dow Corning | 12.10 | 12.10 | 24.20 |
| | Unipure Yellow LC182 | CI 77492 | Sensient | 1.25 | 1.25 | 1.25 |
| | Unipure Red LC381 | CI 77491 | Sensient | 0.45 | 0.45 | 0.45 |
| | Unipure Black LC989 | CI 77499 | Sensient | 0.25 | 0.25 | 0.25 |
| | BLEND 4 | Boron nitride, | Sensient | 12.50 | 15 | 20 |
| | | zinc oxide | | | | |
| F | Dow Corning 245 fluid | Cyclopentasiloxane | Dow Corning | 9.60 | 7.1 | 0 |

Identical formulations were prepared, except that the BLEND 4 whitening composition at 12.5% or at 15% was replaced either by BLEND 8 NEW at 12.5% or at 15% or by BLEND 9 NEW at 12.5% or at 15%.

### 1.2.2.1. Opacity

Table 7 below gives the opacities measured on the cosmetic formulations listed in Table 6.

TABLE 7
Opacity of cosmetic formulations

| | | |
|---|---|---|
| Formula | TiO₂ LC981 (standard) at 10% | 100.46 |
| comprising: | in the absence of whitening | 87.94 |
| | compound (negative bound) | |
| | ZnO (comparative) | 98.6 |
| | BN (comparative) | 97.75 |
| | BLEND 4 FDT at 10% | 98.82 |
| | BLEND 4 FDT at 20% | 99.85 |
| | BLEND 4 FDT at 15% | 100.07 |
| | BLEND 4 FDT at 12.5% | 100.11 |

The results show that the increase in opacifying power with the increase of concentration in BLEND 4 reaches a plateau between 12.5 and 15%.

### 1.2.2.2. LAB Color Measurements on Cup

Table 8 below gives the colorimetric and coverage measurements measured on the cosmetic formulations listed in Table 6.

TABLE 8
Colorimetric measurements (average over three measurements) and coverage
of the different formulas in a L*a*b*CIE 1976 chromatic space.

| | | | | | | | dE/ | dE/ | |
|---|---|---|---|---|---|---|---|---|---|
| | Description | L*(D65) | a*(D65) | b*(D65) | C*(D65) | h(D65) | Bound+ | Bound− | Opacity |
| Formula | LC981 at 10% (standard) | 54.69 | 13.54 | 20.33 | 24.42 | 56.33 | / | / | covering |
| comprising: | in the absence of a whitening | 33.90 | 18.98 | 29.11 | 34.76 | 56.89 | 23.21 | / | not |
| | compound | | | | | | | | covering |
| | (negative bound) | | | | | | | | |
| | BLEND 4 FDT at 10% | 50.50 | 15.21 | 20.89 | 25.84 | 53.94 | 4.55 | 18.90 | covering |
| | BLEND 8 NEW FDT at 10% | 52.65 | 13.78 | 17.79 | 22.50 | 52.24 | 3.26 | 22.51 | covering |
| | BLEND 9 NEW FDT at 10% | 54.23 | 14.95 | 19.95 | 24.93 | 53.15 | 1.53 | 22.66 | covering |
| | BLEND 4 FDT at 12.5 | 55.79 | 13.72 | 18.17 | 22.77 | 52.96 | 2.42 | 25.03 | covering |
| | BLEND 8 NEW FDT at 12.5% | 55.58 | 14.01 | 18.27 | 23.02 | 52.51 | 2.29 | 24.74 | covering |
| | BLEND 9 NEW FDT at 12.5% | 55.44 | 13.55 | 17.71 | 22.30 | 52.59 | 2.72 | 24.96 | covering |
| | BLEND 4 FDT at 15% | 57.59 | 13.95 | 18.21 | 22.94 | 52.54 | 3.61 | 26.56 | covering |
| | BLEND 8 NEW FDT at 15% | 56.20 | 13.40 | 17.36 | 21.93 | 52.34 | 3.34 | 25.82 | covering |
| | BLEND 9 NEW FDT at 15% | 57.01 | 13.38 | 17.55 | 22.07 | 52.70 | 3.62 | 26.44 | covering |
| | BLEND 4 FDT at 20% | 59.55 | 12.35 | 15.87 | 20.11 | 52.12 | 6.70 | 29.62 | covering |

Lightness improves with the increase in the concentration of whitening composition within the cosmetic formulation, but this is not the case for the other colorimetric parameters such as delta E, hence the optimum percentage lies between 12.5 and 15%. Regardless of the percentage, the three BLENDS can be considered as alternatives to titanium dioxide at 80-90% equivalence in the present type of formula environment which is rather a silicone-environment. BLEND 4 FDT gives the best results, especially at 15%.

### 1.3. Preparation of Water-In-Oil Cosmetic Formulations SCT4157 Natural Soy Infused Foundation and Useful as a Foundation

Cosmetic formulations having the compositions shown in detail in Table 9 were prepared.

TABLE 9
Compositions of the water-in-oil cosmetic formulations SCT4157 Natural Soy Infused Foundation
and useful as foundation, the proportions being expressed by weight/weight

| | | | | | | | FDT |
|---|---|---|---|---|---|---|---|
| | | | | | | | formulation |
| | | | | 10% | 12.5% | 15% | (negative |
| Phase | Brand name | INCI | Supplier | formulation | formulation | formulation | bound) |
| A | Symbiomuls WO | Polyglyceryl-3 Polyricinoleate (and) | Dr. | 3.59 | 3.47 | 3.25 | 4.49 |
| | AF | Sorbitan Sesquioleate (and) Cetyl | Straetmans | | | | |
| | | Ricinoleate (and) Glyceryl Caprate | | | | | |
| | | (and) Cera Alba (and) Magnesium | | | | | |
| | | Stearate | | | | | |
| | Jeechem NDA- | C9-12 Alkane | Jeen | 7.18 | 7.0 | 6.51 | 8.97 |
| | LC | | International | | | | |
| | Bentone Gel | Caprylic/Capric Triglyceride (and) | Elementis | 10.26 | 9.93 | 9.30 | 12.82 |
| | GTCC V. | Stearalkonium Hectorite (and) | Specialties | | | | |
| | | Propylene Carbonate | | | | | |
| B | Pure water | Aqua | VWR | 38.52 | 37.30 | 34.9 | 48.15 |
| | Zemea | Propanediol | Dupont Tate | 4.10 | 3.97 | 3.72 | 5.13 |
| | | | & Lyle | | | | |
| | Glycerin | Glycerin | AMI | 1.54 | 1.49 | 1.39 | 1.92 |
| | Euxyl PE 9010 | Phenoxyethanol (and) | Schulke | 0.52 | 0.49 | 0.46 | 0.64 |
| | | Ethylhexylglycerin | | | | | |
| C | Unipure Yellow | CI 77492 | Sensient | 1.25 | 1.24 | 1.16 | 1.60 |
| | LC182 | | | | | | |
| | Unipure Red | CI 77491 | Sensient | 0.46 | 0.45 | 0.42 | 0.58 |
| | LC381 | | | | | | |
| | Unipure Black | CI 77499 | Sensient | 0.26 | 0.25 | 0.23 | 0.32 |
| | LC989 | | | | | | |
| | Natpure FEEL-M | Isopropyl Palmitate (and) Isostearyl | Sensient | 2.06 | 1.98 | 1.86 | 2.56 |
| | ECO | Isostearate (and) Octyldodecanol (and) | | | | | |
| | | Octyldodecyl myristate | | | | | |
| E | Covafluid AMD | Aluminum starch octenylsuccinate | Sensient | 3.08 | 2.98 | 2.79 | 3.85 |
| | HLC talcum | Talcum (and) hydrogenated lecithin | Sensient | 7.18 | 6.95 | 6.51 | 8.97 |
| | Unipure White | IC 77891 (titanium dioxide) or one of | Sensient | 10.00 | 12.5 | 15 | 0 |
| | LC981, one of | the BLENDS of Table 1 | | | | | |
| | the BLENDS | | | | | | |
| | Natpure FEEL-M | Isopropyl Palmitate (and) Isostearyl | Sensient | 10.00 | 10.00 | 12.5 | 0 |
| | ECO | Isostearate (and) Octyldodecanol | | | | | |
| | | (and) | | | | | |
| | | Octyldodecyl myristate | | | | | |

### Procedure:

Prepare Phase A and mix using a Rayneri [mixer] for 10 min at 400 rpm
Grind C with a tricylinder and add to phase A while stirring
Prepare phase B. Slowly add B into A while stirring using a Rayneri [mixer],
Mix between 500 rpm and 800 rpm for 30 min.
Grind the different whites with a tricylindre in the Natpure feel M Eco and add E while stirring to the rest of the formula. Maintain stirring for 10 min at 100 rpm.

Table 10 below gives the viscosities measured on the cosmetic formulations of Table 9.

TABLE 10
Viscosities (CP) measured with a Brookfield viscosimeter
DV2T LVTJO Needle LV4 viscosity SCT4157 TW 1920

| | | BLEND | BLEND | BLEND | | BLEND | BLEND |
|---|---|---|---|---|---|---|---|
| | LC981 | 4 | 8 NEW | 9 NEW | BLEND | 8 NEW | 9 NEW |
| | 10% | 10% | 10% | 10% | 4 12.5% | 12.5% | 12.5% |
| 5 | 37560 | 29040 | 25080 | 30240 | 30840 | 28680 | 31080 |
| 10 | 21420 | 17700 | 15420 | 18300 | 18780 | 17400 | 19320 |
| 20 | 12690 | 10980 | 10050 | 11340 | 12090 | 11100 | 12150 |
| 30 | 9280 | 8340 | 7700 | 8600 | 9300 | 8420 | 9160 |
| 50 | 6324 | 5964 | 5400 | 6216 | 6432 | 5904 | 6552 |
| 80 | 4553 | 4432 | 4050 | 4665 | 4868 | 4425 | 4965 |
| 100 | 3906 | 3882 | 3600 | 4074 | 4302 | 3858 | 4302 |
| 150 | 3032 | 3120 | 2880 | 3260 | 3420 | 3092 | 3456 |
| 200 | 2532 | 2655 | 2469 | 2781 | 2925 | 2649 | 2922 |
| 150 | 2972 | 3088 | 2832 | 3204 | 3388 | 3056 | 3432 |
| 100 | 3834 | 3852 | 3528 | 3972 | 4224 | 3798 | 4254 |
| 80 | 4425 | 4380 | 3997 | 4500 | 4755 | 4357 | 4793 |
| 50 | 6144 | 5748 | 5184 | 5856 | 6336 | 5700 | 6276 |
| 30 | 8920 | 7840 | 7020 | 7880 | 8700 | 7780 | 8340 |
| 20 | 12330 | 9990 | 8970 | 10110 | 11250 | 9540 | 10650 |
| 10 | 21900 | 15960 | 13800 | 15540 | 17940 | 14880 | 17280 |
| 5 | 39960 | 25800 | 21960 | 25560 | 29640 | 26160 | 28920 |

The viscosity of the different BLENDS at 10% and 12.5% remains within the usual range of commercial formulas. The percentage increase does not influence the viscosity of the formulas.

Tables 11 to 13 hereinbelow provide opacity, colorimetric and coverage measurements for the cosmetic formulations listed in Table 9.

TABLE 11
Colorimetric measurements (average over three measurements) in an L*a*b*CIE 1976 chromatic space, opacity and coverage
of cosmetic formulations comprising 10% of TiO2, 10% of whitening composition or free of any whitening compound

| | | | | | | | | | Opacity | |
|---|---|---|---|---|---|---|---|---|---|---|
| | | | | | | | dE/ | dE/ | (ISO | |
| | | L*(D65) | a*(D65) | b*(D65) | C*(D65) | h(D65) | Bound+ | Bound− | 2471) | Coverage |
| Formula | TiO₂ LC981 at 10% (standard) | 58.65 | 12.92 | 19.36 | 23.28 | 56.29 | / | / | 100.7 | covering |
| comprising: | in the absence of whitening compound | 36.59 | 18.08 | 26.64 | 32.20 | 55.8 | 23.80 | / | | not |
| | (negative bound) | | | | | 2 | | | | covering |
| | BLEND 4 FDT at 10% | 49.67 | 15.85 | 23.03 | 27.95 | 55.4 | 10.14 | 13.75 | 88.27 | covering |
| | | | | | | 6 | | | | |
| | BLEND 8 NEW FDT at 10% | 49.12 | 15.99 | 23.05 | 28.05 | 55.2 | 10.68 | 13.19 | 91.05 | covering |
| | | | | | | 5 | | | | |
| | BLEND 9 NEW FDT at 10% | 49.09 | 15.82 | 22.93 | 27.86 | 55.4 | 10.61 | 13.23 | 99.58 | covering |
| | | | | | | 0 | | | | |

TABLE 12
Colorimetric measurements (average over three measurements) in an L*a*b*CIE 1976 chromatic space, opacity and coverage
of cosmetic formulations comprising 12.5% of TiO2, 12.5% of whitening composition or free of any whitening compound

| | | | | | | | | | Opacity | |
|---|---|---|---|---|---|---|---|---|---|---|
| | | | | | | | dE/ | dE/ | (ISO | |
| | | L*(D65) | a*(D65) | b*(D65) | C*(D65) | h(D65) | Bound+ | Bound− | 2471) | Coverage |
| Formula | TiO₂ LC981 at 12.5% | 58.65 | 12.92 | 19.36 | 23.28 | 56.29 | / | / | 100.7 | covering |
| | (standard) | | | | | | | | | |
| comprising: | in the absence of whitening | 36.59 | 18.08 | 26.64 | 32.20 | 55.82 | 23.89 | / | | not |
| | compound (negative bound) | | | | | | | | | covering |
| | BLEND 4 FDT at 12.5% | 51.25 | 15.13 | 21.69 | 26.45 | 55.11 | 8.16 | 15.73 | 98.54 | covering |
| | BLEND 8 NEW FDT at 12.5% | 51.33 | 15.55 | 21.88 | 26.84 | 54.60 | 8.27 | 15.68 | 91.11 | covering |
| | BLEND 9 NEW FDT at 12.5% | 50.87 | 15.35 | 21.82 | 26.68 | 54.88 | 8.61 | 15.30 | 99.21 | covering |

TABLE 13
Colorimetric measurements (average over three measurements) in an L*a*b*CIE 1976 chromatic space, opacity and coverage
of cosmetic formulations comprising 15% of TiO2, 15% of whitening composition or free of any whitening compound

| | | | | | | | | | Opacity | |
|---|---|---|---|---|---|---|---|---|---|---|
| | | | | | | | dE/ | dE/ | (ISO | |
| | | L*(D65) | a*(D65) | b*(D65) | C*(D65) | h(D65) | Bound+ | Bound− | 2471) | Coverage |
| Formula | TiO₂ LC981 at 15% (standard) | 58.65 | 12.92 | 19.36 | 23.28 | 56.29 | / | / | 100.7 | covering |
| comprising | in the absence of whitening compound | 36.59 | 18.08 | 26.64 | 32.20 | 55.82 | 23.80 | / | | not |
| | (negative bound) | | | | | | | | | covering |
| | BLEND 4 FDT at 15% | 53.04 | 13.94 | 19.29 | 23.80 | 54.16 | 5.73 | 18.51 | 97.95 | covering |
| | BLEND 8 NEW FDT at 15% | 52.23 | 14.20 | 19.81 | 24.38 | 54.36 | 6.60 | 17.52 | 99.16 | covering |
| | BLEND 9 NEW FDT at 15% | 52.77 | 13.74 | 19.70 | 24.01 | 55.12 | 5.98 | 18.15 | 105.27 | covering |

At 10% by weight, the results of the different whitening compositions are 50% of the results obtained with 10% titanium dioxide for the formula tested. The BLEND 4 FDT is always the blend with the best results in terms of lightness.

The results improve with the increase in the concentration of whitening composition. At 12.5% by weight, the results of the different whitening compositions are 65% of the results obtained with 10% by weight of titanium dioxide.

Whatever the whitening composition, the best results are obtained at 15% of whitening compositions.

The three BLENDS, used at a concentration of 15% by weight in the formulation, can be considered as replacements for titanium dioxide with a 75% equivalence with respect to a formula comprising 10% by weight of titanium dioxide.

1.4. Preparation of cosmetic oil-in-water formulations SCT2213 Renewed vitality natural BB cream and useful as foundation

In the examples, a cosmetic formulation in the form of a direct oil-in-water emulsion, and of composition shown in detail in Table 14, was used, the latter comprising the whitening compositions listed inf Table 1 in a concentration of 10, 15 or 20% by weight.

TABLE 14
Compositions of the oil-in-water cosmetic formulations SCT2213 Renewed vitality natural
BB cream and useful as foundation, the proportions being expressed by weight/weight

| | | | | | | | | FDT |
|---|---|---|---|---|---|---|---|---|
| | | | | | | | | formulation |
| | | | | | 15% | 20% | 25% | (negative |
| Phase | Brand name | INCI | Supplier | 10% formulation | formulation | formulation | formulation | bound) |
| A | Pure water | Aqua | VWR | 40.67 | 40.67 | 38.78 | 33.78 | 47.6 |
| | Glycerin | Glycerin | AMI | 3.51 | 3.51 | 3.34 | 2.91 | 4.10 |
| | Thickagent LC | Xanthan gum (and) | Sensient | 0.35 | 0.35 | 0.33 | 0.3 | 0.41 |
| | | Hectorite (and) | | | | | | |
| | | Cellulose | | | | | | |
| | Microcare PE | Phenoxyethanol | Thor | 0.7 | 0.7 | 0.67 | 0.58 | 0.82 |
| B | Emulium Mellifera | Polyglyceryl-6 | Gattefosse | 2.8 | 2.8 | 2.68 | 2.33 | 3.29 |
| | MB | Distearate (and) Jojoba | | | | | | |
| | | Esters (and) | | | | | | |
| | | Polyglyceryl-3 Beeswax | | | | | | |
| | | (and) Cetyl Alcohol | | | | | | |
| | Natpure FEEL-M | Isopropyl Palmitate | Sensient | 7.01 | 7.01 | 6.69 | 5.82 | 8.20 |
| | ECO | (and) Isostearyl | | | | | | |
| | | Isostearate (and) | | | | | | |
| | | Octyldodecanol (and) | | | | | | |
| | | Octyldodecyl myristate | | | | | | |
| | Lipex Shea | Butyrospermum Parkii | AAK | 3.51 | 3.51 | 3.34 | 2.91 | 4.10 |
| | | (Shea) Butter | | | | | | |
| | Miglyol 812 | Caprylic/capric | Unipex | 3.51 | 3.51 | 3.34 | 2.91 | 4.10 |
| | | triglyceride | | | | | | |
| C | Covarine Red WN | CI 77491 (and) Glycerin | Sensient | 1.06 | 1.06 | 1.02 | 0.88 | 1.24 |
| | 3798 | (and) Aqua (and) | | | | | | |
| | | Xanthan Gum (and) | | | | | | |
| | | Sodium Citrate (Citric | | | | | | |
| | | Acid) | | | | | | |
| | Covarine Yellow | CI 77492 (and) Glycerin | Sensient | 2.37 | 2.37 | 2.28 | 1.98 | 2.79 |
| | WN 1798 | (and) Aqua (and) | | | | | | |
| | | Xanthan Gum (and) | | | | | | |
| | | Sodium Citrate (Citric | | | | | | |
| | | Acid) | | | | | | |
| | Covarine Black | CI 77499 (and) Glycerin | Sensient | 0.53 | 0.53 | 0.51 | 0.44 | 0.62 |
| | WN 9798 | (and) Aqua (and) | | | | | | |
| | | Xanthan Gum (and) | | | | | | |
| | | Sodium Citrate (Citric | | | | | | |
| | | Acid) | | | | | | |
| D | Glycerin | Glycerin | | 23.98 | 18.98 | 17.02 | 20.16 | 22.73 |
| | Unipure White | CI 77891 (Titanium | Sensient | 10.00 | 15.00 | 20.00 | 25.00 | 0 |
| | LC981, one of the | dioxide), one of the | | | | | | |
| | BLENDS | BLENDS | | | | | | |

### Procedure:

Pre-mix the Thickagent LC into glycerine and while stirring, incorporate same into the rest of phase A.
Prepare and heat phase B to 75° C. Add phase B to phase A while stirring.
Add phase C.
Grind the different whites, with a tricylinder, in glycerine. Homogenize the whole under an IKA shaker.

Table 15 below gives the viscosities measured on the cosmetic formulations of Table 14.

TABLE 15
Viscosities (CP) measured with a Brookfield viscosimeter
DV2T LVTJO Needle LV4 viscosity SCT2213 TW 1920

| | | BLEND | BLEND | BLEND | BLEND | BLEND | BLEND | BLEND |
|---|---|---|---|---|---|---|---|---|
| Speed | LC981 | 4 | 8 NEW | 9 NEW | 4 | 4 | 8 NEW | 9 NEW |
| (rpm) | 10% | 15% | 15% | 15% | 20% | 25% | 25% | 25% |
| 5 | 27360 | 40800 | 38400 | 36360 | 67920 | 42240 | 51720 | 73320 |
| 10 | 15780 | 22860 | 21180 | 20040 | 37620 | 35520 | 28080 | 38940 |
| 20 | 9720 | 12300 | 11580 | 11100 | 19710 | 24810 | 15300 | 21150 |
| 30 | 6780 | 8660 | 8160 | 8120 | 13800 | 16920 | 10860 | 14780 |
| 50 | 4656 | 5700 | 5340 | 5352 | 9060 | 10510 | 7092 | 9432 |
| 80 | 3278 | 3825 | 3720 | 3735 | 6053 | — | 4860 | 6427 |
| 100 | 2736 | 3156 | 3090 | 3132 | 5004 | — | 4068 | 5364 |
| 150 | 1956 | 2264 | 2216 | 2300 | 3632 | — | 2972 | 3884 |
| 200 | 1569 | 1758 | 1725 | 1821 | 2877 | — | 2382 | — |
| 150 | 1856 | 2184 | 2104 | 2224 | 3544 | — | 2864 | 3768 |
| 100 | 2484 | 2994 | 2850 | 3042 | 4884 | 5838 | 3828 | 5040 |
| 80 | 2963 | 3615 | 3472 | 3653 | 5865 | 6390 | 4560 | 5775 |
| 50 | 4284 | 5316 | 5064 | 5304 | 8568 | 7824 | 6576 | 8364 |
| 30 | 6160 | 7920 | 7700 | 8060 | 12800 | 8620 | 9840 | 12380 |
| 20 | 8280 | 11310 | 10860 | 11430 | 17640 | 9420 | 13590 | 16800 |
| 10 | 13800 | 20760 | 19560 | 20880 | 30780 | 10140 | 24000 | 29880 |
| 5 | 23400 | 37800 | 35400 | 37560 | 53640 | 12360 | 42480 | 54120 |

The viscosity of the different BLENDS remains within the usual range of commercial formulas. The percentage increase does not influence the viscosity of the formulas.

TABLE 16
Colorimetric measurements (average over three measurements) in an L*a*b*CIE 1976 chromatic space, opacity and coverage of
cosmetic formulations comprising 10% of TiO₂, 10, 15 or 20% of whitening composition or free of any whitening compound

| | | | | | | | | | Opacity | |
|---|---|---|---|---|---|---|---|---|---|---|
| | | | | | | | dE/ | dE/ | (ISO | |
| | | L*(D65) | a*(D65) | b*(D65) | C*(D65) | h(D65) | Bound+ | Bound− | 2471) | Coverage |
| Formula | TiO₂ LC981 at 10% | 57.33 | 14.02 | 19.57 | 24.07 | 54.40 | / | / | 100.58 | covering |
| comprising: | (standard) | | | | | | | | | |
| | in the absence of whitening | 39.65 | 18.57 | 24.59 | 30.81 | 52.94 | 18.93 | / | 95.8 | not |
| | compound (negative bound) | | | | | | | | | covering |
| | BLEND 4 FDT at 10% | 48.95 | 16.46 | 21.54 | 27.11 | 52.61 | 8.94 | 9.87 | 98.91 | covering |
| | BLEND 8 NEW FDT at 10% | 48.25 | 16.73 | 22.21 | 27.80 | 53.01 | 9.83 | 8.97 | 102.31 | covering |
| | BLEND 9 NEW FDT at 10% | 46.82 | 17.27 | 22.95 | 28.72 | 53.04 | 11.50 | 7.33 | 95.9 | covering |
| | BLEND 4 FDT at 15% | 52.78 | 15.76 | 20.54 | 25.89 | 52.50 | 4.97 | 13.88 | 99.94 | covering |
| | BLEND 8 NEW FDT at 15% | 52.45 | 15.65 | 20.68 | 25.93 | 52.89 | 5.25 | 13.56 | 99.42 | covering |
| | BLEND 9 NEW FDT at 15% | 52.33 | 15.79 | 20.95 | 26.23 | 52.99 | 5.48 | 13.34 | 100.94 | covering |
| | BLEND 4 FDT at 20% | 54.95 | 15.05 | 19.93 | 24.98 | 52.95 | 2.62 | 16.24 | 105.09 | covering |
| | BLEND 4 FDT at 25% | 58.18 | 13.87 | 18.19 | 22.87 | 52.67 | 1.62 | 20.02 | 103.84 | covering |
| | BLEND 8 NEW FDT at 25% | 56.71 | 14.15 | 17.78 | 22.72 | 51.46 | 1.88 | 18.75 | 101.66 | covering |
| | BLEND 9 NEW FDT at 25% | 58.08 | 13.90 | 17.88 | 22.65 | 52.14 | 1.84 | 20.02 | 103.96 | covering |

Table 16 hereinabove provides the opacity, colorimetric and coverage measurements for the cosmetic formulations listed in Table 14.

At 10% by weight, the results of the different whitening compositions are 50% of the results obtained with 10% titanium dioxide, for said formula as well.

Whatever the whitening composition, the best results are obtained at 25% of whitening composition. In terms of opacity and lightness, the results of the three BLENDS 4, 8 NEW and 9 NEW are at the same level as the results of the 10% titanium dioxide formula.

The three BLENDS used at a concentration of 25% by weight in the formulation can be considered as replacements for titanium dioxide with respect to a formula comprising 10% by weight of titanium dioxide.

Regardless of the formulation environment (examples 1.2, 1.3 and 1.4 hereinabove), the three BLENDS 4, 8 NEW and 9 NEW are good alternatives to titanium dioxide.

### Example 2: Whitening Compositions for Lipstick

### 2.1. Preparation of Whitening Compositions

Since commercial formulas are cheaper and require less opacity and hue lightening than foundations, the percentage of boron nitride in the whitening compositions has been reduced.

The whitening compositions having the composition detailed in Table 17 were prepared.

TABLE 17
Compositions of the whitening compositions, the proportions being expressed by weight/weight

| | | BLEND | BLEND | | BLEND | | | BLEND | |
|---|---|---|---|---|---|---|---|---|---|
| | BLEND | 7 | 8 | BLEND | 9 | BLEND | BLEND | 11 | BLEND |
| | 3 | RAL | RAL | 9 | NEW | 10 | 11 | NEW | 12 |
| | RAL | (comparative) | (comparative) | RAL | RAL | RAL | RAL | RAL | RAL |
| Boron | 27 | 5 | 10 | 28 | 22 | 23 | 26 | 21 | 18 |
| nitride | | | | | | | | | |
| Zinc oxide | 73 | 90 | 81 | 52 | 73 | 57 | 54 | 74 | 62 |
| Bismuth | — | 5 | — | — | — | — | — | — | — |
| oxychloride | | | | | | | | | |
| Cerium | — | — | 9 | — | — | — | — | — | — |
| oxide | | | | | | | | | |
| Kaolin | — | — | — | 20 | 5 | — | — | — | — |
| Magnesium | — | — | — | — | — | 20 | — | — | — |
| oxide | | | | | | | | | |
| Tricalcium | — | — | — | — | — | — | 20 | 5 | — |
| citrate | | | | | | | | | |
| Mixture of | — | — | — | — | — | — | — | — | 20 |
| hydroxyapa | | | | | | | | | |
| tite and | | | | | | | | | |
| calcium | | | | | | | | | |
| carbonate | | | | | | | | | |
| ZnO/BN | 2.70 | 18 | 8.10 | 1.86 | 3.32 | 2.49 | 2.08 | 3.52 | 3.44 |
| ratio | | | | | | | | | |

### Procedure for Making Each BLEND:

Mixing using a commercial Waring spice mill from Cuisinart PRO-PREP CHOPPER GRINDER CHOP tank with a stirring of 2×15 s (scraping between 2 turns).
   1.2. Preparation of Cosmetic Formulations with Carmin and Useful as Lipstick

Each whitening composition listed in Table 17 was tested at a concentration of 16%, 20% or 25% by weight in a formula comprising 4% by weight of Carmin. Cosmetic formulations having the compositions shown in detail in Table 18 were prepared.

TABLE 18
Compositions of the cosmetic formulations comprising 4% by weight of Carmin
and useful as lipstick, the proportions being expressed by weight/weight

| | | | | | | | RAL |
|---|---|---|---|---|---|---|---|
| | | | | | | | formulation |
| | | | | 16% | 20% | 25% | (negative |
| Phase | Brand name | INCI | Supplier | formula | formula | formula | bound) |
| A | Covalip 94 WP | Paraffin (and) Isopropyl palmitate (and) | Sensient | 31.00 | 31.00 | 31.00 | 31.00 |
| | | Ozokerite (and) Paraffinum Liquidum (and) | | | | | |
| | | Copernicia Cerifera Cera (and) Candelilla | | | | | |
| | | Cera (and) Butyl stearate (and) Ethylene/VA | | | | | |
| | | copolymer (and) Benzyl alcohol (and) | | | | | |
| | | Dehydroacetic acid | | | | | |
| | Beaver oil | Ricinus communis (beaver) seed oil | Mosselman | 25.00 | 25.00 | 25.00 | 25.00 |
| B | Ariabel Red Carmine | CI75470 (and) Maltodextrin | Sensient | 4.00 | 4.00 | 4.00 | 4.00 |
| | ST358 | | | | | | |
| | Unipure White LC981, | CI 77891 (titanium dioxide), boron nitride, zinc | Sensient | 16.00 | 20.00 | 25.00 | 0 |
| | or Caress BN02, or zinc | oxide, one of the BLENDs listed in Table 17 | | | | | |
| | oxide, or one of the | | | | | | |
| | BLENDs listed in table | | | | | | |
| | 17 | | | | | | |
| | Crodamol IPP | Isopropyl Palmitate | Croda | 24.00 | 20.00 | 15.00 | 40.00 |

### Procedure:

Melt Phase A in a water bath
Grind phase B using a tricylinder, 3 passes
Place B in A while stirring in a water bath.

Table 19 hereinbelow provides the opacity, colorimetric and coverage measurements for the cosmetic formulations listed in Table 18.

TABLE 19
Colorimetric measurements (average over three measurements) in an L*a*b*CIE 1976 chromatic space,
opacity and coverage of cosmetic formulations comprising 16% of TiO₂, of boron nitride (alone) or
of zinc oxide (alone), 16, 20 or 25% of whitening composition or free of any whitening compound

| | | | | | | | dE/ | dE | | | |
|---|---|---|---|---|---|---|---|---|---|---|---|
| | | L*(D65) | a*(D65) | b*(D65) | C*(D65) | h(D65) | Bound+ | Bound− | dL | Opacity | Coverage |
| Formula | TiO₂ LC981 at 16% | 50.32 | 33.85 | −0.44 | 33.86 | 359.26 | / | / | / | 96.21 | covering |
| at 16% | BLEND 3 RAL at 16% | 35.69 | 39.96 | −0.21 | 39.96 | 359.70 | 15.86 | 26.16 | −14.63 | 69.7 | covering |
| | BLEND 7 at 16% | 33.98 | 40.05 | −1.27 | 40.07 | 358.18 | 17.49 | 25.46 | −16.34 | 54.76 | moderately |
| | | | | | | | | | | | covering |
| | BLEND 8 at 16% | 34.55 | 39.76 | −0.53 | 39.76 | 359.24 | 16.85 | 25.37 | −15.78 | 64.37 | moderately |
| | | | | | | | | | | | covering |
| | BLEND 9 at 16% | 34.48 | 39.09 | 0.94 | 39.10 | 1.37 | 16.74 | 24.31 | −15.85 | 56.26 | covering |
| | BLEND 9 NEW at 16% | 43.18 | 27.13 | −2.55 | 27.24 | 354.61 | 10.04 | 15.05 | −7.14 | 83.47 | covering |
| | BLEND10 at 16% | 34.34 | 40.52 | 1.16 | 40.54 | 1.64 | 17.39 | 24.62 | −15.98 | 49.76 | moderately |
| | | | | | | | | | | | covering |
| | BLEND 11 at 16% | 34.20 | 39.54 | 1.21 | 39.56 | 1.76 | 17.18 | 24.11 | −16.13 | 62.88 | covering |
| | BLEND 11 NEW at 16% | 43.37 | 26.65 | −2.93 | 26.81 | 353.73 | 10.31 | 15.60 | −6.95 | 70.67 | covering |
| | BLEND12 at 16% | 33.34 | 40.49 | 0.59 | 40.49 | 0.83 | 17.18 | 24.13 | −16.99 | 46.8 | moderately |
| | | | | | | | | | | | covering |
| | in the absence of | 15.18 | 30.85 | 13.23 | 33.57 | 23.22 | 37.82 | / | −35.14 | 15.88 | not |
| | any whitening compound | | | | | | | | | | covering |
| | (negative bound) | | | | | | | | | | |
| | Boron nitride | 38.53 | 39.51 | 3.84 | 39.70 | 5.55 | 13.76 | 26.62 | −11.79 | 74.34 | covering |
| | alone at 16% (comparative) | | | | | | | | | | |
| | Zinc oxide | 34.47 | 40.51 | −1.67 | 40.55 | 357.63 | 17.23 | 26.22 | −15.85 | 47.04 | moderately |
| | alone at 16% (comparative) | | | | | | | | | | covering |
| Formula | BLEND 3 RAL at 20% | 38.00 | 39.32 | −1.14 | 39.33 | 358.35 | 13.50 | 28.26 | −12.32 | 88.48 | covering |
| at 20% | BLEND 7 at 20% | 37.12 | 39.67 | −2.15 | 39.73 | 356.90 | 14.53 | 28.20 | −13.21 | 69.62 | covering |
| | BLEND 8 at 20% | 37.89 | 36.91 | −1.33 | 36.93 | 357.96 | 12.83 | 27.65 | −12.43 | 77.36 | covering |
| | BLEND 9 at 20% | 36.43 | 39.18 | −0.34 | 39.18 | 359.49 | 14.88 | 26.55 | −13.90 | 83.17 | covering |
| | BLEND 11 at 20% | 36.39 | 39.67 | 0.14 | 39.67 | 0.19 | 15.11 | 26.44 | −13.93 | 87.13 | covering |
| Formula | BLEND 3 RAL at 25% | 40.35 | 39.01 | −2.05 | 39.06 | 356.99 | 11.35 | 30.55 | −9.98 | 82.39 | covering |
| at 25% | BLEND 7 at 25% | 39.23 | 39.38 | −3.02 | 39.49 | 355.61 | 12.66 | 30.25 | −11.09 | 71.62 | covering |
| | BLEND 8 at 25% | 39.65 | 39.04 | −2.33 | 39.11 | 356.58 | 12.02 | 30.13 | 10.67 | 77.95 | covering |
| | BLEND 9 at 25% | 38.78 | 39.71 | −0.68 | 39.71 | 359.02 | 12.95 | 28.78 | 11.55 | 81.04 | covering |
| | BLEND 11 at 25% | 38.79 | 39.57 | −0.74 | 39.58 | 358.92 | 12.88 | 28.78 | 11.54 | 82.61 | covering |

For lipsticks, the most determining parameter is opacity, colorimetric measurements comprising the tint (a*, b*) and the lightness (L*) should also be taken into account as a whole.

The positive bound (standard) is the formula with 16% anatase titanium dioxide and the negative bound is the formula free of any whitening compound.

BLENDS 10 and 12 give poor opacity results. Magnesium oxide and the mixture of hydroxyapatite and calcium carbonate do not appear to be suitable whitening compounds.

Boron nitride used alone gives very good opacity results. With the naked eye and according to colorimetric measurements, the formula with boron nitride has a yellow hue and that with zinc oxide has a blue hue. Advantageously, the BLEND 3 at 16% imparts to the formulation containing a neutral tint (neither yellow nor blue). The above observation is confirmed by the results for the parameter b*. Negative values of said parameter indicate an increasingly blue hue and positive values an increasingly yellow hue. Thereby, boron nitride neutralizes the blue hue of zinc oxide and zinc oxide neutralizes the yellow hue of boron nitride.

Optimization tests for BLEND 9 and BLEND 11 closer to the total ZnO+BN concentration of the BLEND 3 composition were carried out with BLEND 9 NEW and BLEND 11 NEW in order to further neutralize the yellowish side given by boron nitride. In terms of opacity and lightness, BLEND 9 NEW and 11 NEW give a better performance, but on the other hand the hue of the formulations obtained is far too blue (parameter b). Having a balanced hue in the yellow and blue tint is important for lipstick application. A ratio between the weight of zinc oxide and the weight of boron nitride of less than 3.0 is thus preferred.

The BLEND 9 NEW and 11 NEW being eliminated from the tests, BLEND 3 is the blend which gives the best results in terms of opacity and of lightness. BLEND 3, used at a concentration of 16% by weight in the formulation, can be considered as a replacement for titanium dioxide with a 50% equivalence with respect to a formula comprising 16% by weight of titanium dioxide.

The tests at 20% and 25% were carried out for BLENDS 3,7,8,9 and 11. The positive bound remains the formula with titanium dioxide at 16%. Increasing the concentration of whitening composition improves the results of the different parameters. BLENDS 7 and 8 do not reach the level of opacity of the other 3 BLENDS so same will no longer be considered as high-performance whitening compositions. Between 20 and 25% of whitening composition, the opacity results reach a plateau. BLENDS 3, 9 and 11 are all three good replacement candidates for titanium dioxide with an equivalence of 50 to 75% depending on the concentration of whitening composition within the cosmetic formulation with respect to the formula with titanium dioxide at 16%. BLEND 3 is the blend which gives the best results to the formula in terms of opacity and of lightness, reaching up to 75% equivalence compared to the formula with 16% titanium dioxide.

For the rest of the tests, the concentration of whitening composition in cosmetic formulations is 16%, in order to optimize the costs for lipstick formulas.

1.3. Preparation of Cosmetic Formulations with Red 7 Toner and Useful as Lipstick

Each whitening composition listed in Table 17 was tested at a concentration of 16% by weight in a formula comprising a pigment other than carmine, red 7 toner (pigment concentration >90%) known by the name Unipure Red LC3079 at 4% by weight, average percentage for use in a lipstick.

Cosmetic formulations having the compositions shown in detail in Table 20 were prepared.

TABLE 20
Compositions of the cosmetic formulations comprising 4% by weight of Red 7 toner and useful
as lipstick, the proportions being expressed by weight/weight

| | | | | | RAL |
|---|---|---|---|---|---|
| | | | | | formulation |
| | | | | 16% | (negative |
| Phase | Brand name | INCI | Supplier | formula | bound) |
| A | Covalip 94 WP | Paraffin (and) Isopropyl palmitate | Sensient | 31.00 | 31.00 |
| | | (and) Ozokerite (and) | | | |
| | | Paraffinum Liquidum (and) | | | |
| | | Copernicia Cerifera Cera (and) | | | |
| | | Candelilla Cera (and) Butyl | | | |
| | | stearate (and) Ethylene/VA | | | |
| | | copolymer (and) Benzyl alcohol | | | |
| | | (and) Dehydroacetic acid | | | |
| | Beaver oil | Ricinus communis (beaver) seed oil | Mosselman | 25.00 | 25.00 |
| B | Unipure Red LC3079 | CI 15850 | Sensient | 4.00 | 4.00 |
| | Unipure White LC981, or | CI 77891 (titanium dioxide), | Sensient | 16.00 | 0 |
| | Caress BN02, or zinc oxide, | boron nitride, zinc oxide, one | | | |
| | or one of the BLENDs listed | of the BLENDs listed in Table 17 | | | |
| | in table 17 | | | | |
| | Crodamol IPP | Isopropyl Palmitate | Croda | 24.00 | 40.00 |

Table 21 hereinbelow provides the opacity, colorimetric and coverage measurements for the cosmetic formulations listed in Table 20.

TABLE 21
Colorimetric measurements (average over three measurements) in an L*a*b*CIE 1976 chromatic space, opacity and coverage
of cosmetic formulations comprising 16% of TiO2 of whitening composition or free of any whitening compound

| | | | | | | | dE/ | dE/ | | | |
|---|---|---|---|---|---|---|---|---|---|---|---|
| | | L*(D65) | a*(D65) | b*(D65) | C*(D65) | h(D65) | Bound+ | Bound− | dL | Opacity | Coverage |
| Formula | TiO₂ LC981 at 16% | 35.90 | 52.54 | 12.24 | 53.95 | 13.12 | / | / | / | 90.00 | covering |
| at 16% | (standard) | | | | | | | | | | |
| | BLEND 3 RAL at 16% | 33.36 | 52.38 | 21.70 | 56.92 | 21.70 | 8.96 | 15.27 | −2.54 | 87.45 | covering |
| | BLEND 7 at 16% | 32.03 | 53.01 | 22.33 | 57.52 | 22.84 | 10.82 | 13.51 | −3.87 | 78.49 | covering |
| | BLEND 8 at 16% | 32.32 | 52.50 | 21.76 | 56.84 | 22.51 | 10.17 | 14.05 | −3.58 | 77.01 | covering |
| | BLEND 9 at 16% | 32.79 | 52.24 | 22.48 | 56.87 | 23.28 | 10.70 | 13.53 | −3.11 | 79.28 | covering |
| | BLEND 9 NEW at 16% | 39.88 | 40.45 | 12.64 | 42.38 | 17.35 | 12.74 | 27.28 | 3.98 | 72.38 | covering |
| | BLEND 11 at 16% | 32.28 | 52.13 | 22.80 | 56.90 | 23.62 | 11.17 | 13.03 | −3.62 | 85.38 | covering |
| | BLEND 11 NEW at 16% | 39.04 | 42.00 | 13.74 | 44.19 | 18.10 | 11.11 | 25.46 | 3.14 | 76.43 | covering |
| | in the absence of | 27.55 | 50.47 | 34.82 | 61.32 | 34.60 | 24.16 | / | −8.35 | 76.41 | not |
| | any whitening compound | | | | | | | | | | covering |
| | (negative bound) | | | | | | | | | | |

For a formula comprising red 7 toner as pigment, the phenomenon contrary to the phenomenon occurring with the formula comprising carmine is observed: BLENDS 9 and 11 impart more opacifying properties to the formulation comprising same than BLENDS 9 NEW and 11 NEW. Same have also a better performance than BLENDS 7 and 8.

Regarding the blue hue observed with carmine for BLENDS 9 NEW and 11 NEW, same does not have the same extent for the formula including Red 7 Toner, on the other hand, a green halo is visible.

In terms of opacity and lightness, BLEND 3 is always the blend which imparts the best results to the formula

Taking into account colorimetric, opacity and coverage measurements and observations with the naked eye, BLEND 3, used at a concentration of 16% by weight in the formulation, can be considered as a replacement for titanium dioxide with a 60% equivalence with respect to a formula comprising 16% by weight of titanium dioxide. After BLEND 3, BLEND 9 and then BLEND 11 have a good performance as well.

The difference between the formula with titanium dioxide and without titanium dioxide is reduced compared to the formula using carmine.

1.4. Preparation of Cosmetic Formulations with Another Pigment and Useful as Lipstick

For the rest of the tests, BLENDS 3, 9 and 11 listed in Table 17 were kept by making formulas comprising 16%, but with pigments from other families than carmine and red 7 Toner and commonly used in lipsticks:
Red 7 lake (30-50% concentration of pure coloring agent) known as Unipure Red LC3075 at 4% in the formula like carmine, average percentage for use in a lipstick
Blue 1 lake (10-20% concentration of coloring agent) known as Unipure Blue LC621 at 1%, average percentage for use in a lipstick use for tint adjustments.
Yellow 5 lake (20-35% concentration of coloring agent) known as Unipure Yellow LC 125 at 2%, average percentage for use in a lipstick as well because same is used in very small quantities for tint adjustments.
Yellow iron oxide known as Unipure Yellow LC182 at 1%, average percentage for use in a lipstick for tint adjustments.
Black iron oxide known as Unipure Black LC 989 at 1%, average percentage of use in lipstick or use in a lipstick for tint adjustments.

Cosmetic formulations having the compositions shown in detail in Table 22 were prepared.

TABLE 22
Compositions of cosmetic formulations comprising another pigment and useful as lipstick, the proportions being expressed by weight/weight. Tables 23 to 27
hereinbelow (a table of results per pigment tested) provide the opacity and colorimetric measurements for the cosmetic formulations listed in Table 22.

| | | | | | | | | | | | Yellow | | Iron |
|---|---|---|---|---|---|---|---|---|---|---|---|---|---|
| | | | | | Red 7 | | Blue 1 | | Yellow 5 | 16% | iron | 16% | oxide |
| | | | | Formula | lake | 16% | lake | 16% | lake | yellow | oxide | black | black |
| | | | | Red | formula | Blue 1 | formula | Yellow | formula | iron | formula | iron | formula |
| | Brand | | | 7 lake at | (negative | lake | (negative | 5 lake | (negative | oxide | (negative | oxide | (negative |
| Phase | name | INCI | Supplier | 16% | bound) | formula | bound) | formula | bound) | formula | bound) | formula | bound) |
| A | Covalip 94 | Paraffin (and) | Sensient | 31.00 | 31.00 | 31.00 | 31.00 | 31.00 | 31.00 | 31.00 | 31.00 | 31.00 | 31.00 |
| | WP | Isopropyl palmitate | | | | | | | | | | | |
| | | (and) Ozokerite | | | | | | | | | | | |
| | | (and) Paraffinum | | | | | | | | | | | |
| | | Liquidum (and) | | | | | | | | | | | |
| | | Copernicia Cerifera | | | | | | | | | | | |
| | | Cera (and) Candelilla | | | | | | | | | | | |
| | | Cera (and) Butyl | | | | | | | | | | | |
| | | stearate (and) | | | | | | | | | | | |
| | | Ethylene/VA copolymer | | | | | | | | | | | |
| | | (and) Benzyl alcohol | | | | | | | | | | | |
| | | (and) Dehydroacetic | | | | | | | | | | | |
| | | acid | | | | | | | | | | | |
| | Beaver oil | Ricinus communis | Mosselman | 25.00 | 25.00 | 25.00 | 25.00 | 25.00 | 25.00 | 25.00 | 25.00 | 25.00 | 25.00 |
| | | (beaver) seed oil | | | | | | | | | | | |
| B | Unipure | CI 15850 (and) | Sensient | 4.00 | 4.00 | 0 | 0 | 0 | 0 | 0 | 0 | 0 | 0 |
| | Red | Aluminum Hydroxide | | | | | | | | | | | |
| | LC3075 | | | | | | | | | | | | |
| | Unipure | CI 42090 | Sensient | 0 | 0 | 1.00 | 1.00 | 0 | 0 | 0 | 0 | 0 | 0 |
| | Blue | | | | | | | | | | | | |
| | LC621 | | | | | | | | | | | | |
| | Unipure | CI 19140 | Sensient | 0 | 0 | 0 | 0 | 2.00 | 2.00 | 0 | 0 | 0 | 0 |
| | Yellow | | | | | | | | | | | | |
| | LC125 | | | | | | | | | | | | |
| | Unipure | CI 77492 | Sensient | 0 | 0 | 0 | 0 | 0 | 0 | 1.00 | 1.00 | 0 | 0 |
| | Yellow | | | | | | | | | | | | |
| | LC182 | | | | | | | | | | | | |
| | Unipure | CI77499 | Sensient | 0 | 0 | 0 | 0 | 0 | 0 | 0 | 0 | 1.00 | 1.00 |
| | Black | | | | | | | | | | | | |
| | LC989 | | | | | | | | | | | | |
| | Unipure | CI 77891 | Sensient | 16.00 | 0 | 16.00 | 0 | 16.00 | 0 | 16.00 | 0 | 16.00 | 0 |
| | White | (titanium | | | | | | | | | | | |
| | LC981, or | dioxide), | | | | | | | | | | | |
| | Caress | boron nitride, | | | | | | | | | | | |
| | BN02, or | zinc oxide, | | | | | | | | | | | |
| | zinc oxide, | one of the | | | | | | | | | | | |
| | or one of | BLENDs | | | | | | | | | | | |
| | BLENDS | listed in | | | | | | | | | | | |
| | the | Table 17 | | | | | | | | | | | |
| | listed in | | | | | | | | | | | | |
| | table 17 | | | | | | | | | | | | |
| | Crodamol | Isopropyl | Croda | 24.00 | 40.00 | 27.00 | 43.00 | 26.00 | 42.00 | 27.00 | 43.00 | 27.00 | 43.00 |
| | IPP | Palmitate | | | | | | | | | | | |

TABLE 23
Colorimetric measurements (average over three measurements) in an L*a*b*CIE 1976 chromatic space
and opacity of cosmetic formulations comprising Red 7 lake as piament and useful as lipstick

| | | | | | | | dE/ | dE/ | | |
|---|---|---|---|---|---|---|---|---|---|---|
| | | L*(D65) | a*(D65) | b*(D65) | C*(D65) | h(D65) | Bound+ | Bound− | dL | Opacity |
| Formula | TiO₂ LC981 at 16% (standard) | 51.19 | 43.59 | 2.29 | 43.66 | 3.01 | / | / | / | 86.80 |
| at 16% | in the absence of whitening compound | 38.35 | 39.52 | 16.97 | 43.01 | 23.24 | 19.93 | / | −12.85 | 28.13 |
| | (negative bound) | | | | | | | | | |
| | BLEND 3 RAL at 16% | 37.71 | 52.81 | 8.19 | 53.45 | 8.82 | 17.37 | 15.94 | 13.48 | 64.13 |
| | BLEND 9 at 16% | 36.94 | 53.45 | 8.55 | 54.12 | 9.09 | 18.42 | 16.33 | −14.25 | 55.96 |
| | BLEND 11 at 16% | 36.94 | 53.05 | 10.26 | 54.04 | 10.94 | 18.87 | 15.17 | −14.26 | 50.76 |

All the 16% formulas listed in Table 23 are covering.

For Red 7 lake, the 3 BLENDS 3, 9 and 11, used at a concentration of 16%, impart very good performance to the formulation in terms of opacity, with results at 80% compared with the results obtained with the formula comprising 16% titanium dioxide. However, in terms of tint, the performance is lower than the performance observed for formulas containing carmine or Red 7 Toner.

TABLE 24
Colorimetric measurements (average over three measurements) in an L*a*b*CIE 1976 chromatic space
and opacity of cosmetic formulations comprising Blue1 lake as pigment and useful as lipstick

| | | | | | | | dE/ | dE/ | | |
|---|---|---|---|---|---|---|---|---|---|---|
| | | L*(D65) | a*(D65) | b*(D65) | C*(D65) | h(D65) | Bound+ | Bound− | dL | Opacity |
| Formula | TiO₂ LC981 at 16% (standard) | 71.96 | −17.34 | −17.99 | 24.98 | 226.05 | | / | / | 78.74 |
| at 16% | in the absence of whitening compound | 33.86 | −6.07 | −20.49 | 21.37 | 253.50 | −38.10 | 39.81 | / | 9.29 |
| | (negative bound) | | | | | | | | | |
| | BLEND 3 RAL at 16% | 61.61 | −20.06 | −26.14 | 32.96 | 232.49 | −10.35 | 13.45 | 31.60 | 62.13 |
| | BLEND 9 at 16% | 61.61 | 19.63 | −25.87 | 32.48 | 232.81 | −10.35 | 13.21 | 31.36 | 58.76 |
| | BLEND 11 at 16% | 61.43 | 19.76 | −25.89 | 32.57 | 232.65 | −10.53 | 13.38 | 31.26 | 47.28 |

All the 16% formulas listed in Table 24 are covering.

BLEND 3 stands out with a better result imparted to the formula which contains same in terms of opacity, which is confirmed with observations with the naked eye. For the rest of the measurements, the 3 BLENDS tested give very similar results. The blends are good replacements for titanium dioxide, with 70% equivalence with respect to the formula comprising 16% titanium dioxide.

TABLE 25
Colorimetric measurements (average over three measurements) in an L*a*b*CIE 1976 chromatic space
and opacity of cosmetic formulations comprising Yellow 5 lake as pigment and useful as lipstick

| | | | | | | | dE/ | dE/ | | |
|---|---|---|---|---|---|---|---|---|---|---|
| | | L*(D65) | a*(D65) | b*(D65) | C*(D65) | h(D65) | Bound+ | Bound− | dL | Opacity |
| Formula | TiO₂ LC981 at 16% (standard) | 84.61 | 11.32 | 61.28 | 62.32 | 79.53 | / | / | / | 45.98 |
| at 16% | in the absence of whitening compound | 65.59 | 27.12 | 65.56 | 70.95 | 67.54 | 25.10 | / | −19.03 | 6.74 |
| | (negative bound) | | | | | | | | | |
| | BLEND 3 RAL at 16% | 82.21 | 14.86 | 67.74 | 69.35 | 77.62 | 7.75 | 20.77 | −2.41 | 51.89 |
| | BLEND 9 at 16% | 82.12 | 15.66 | 68.98 | 70.73 | 77.21 | 9.18 | 20.41 | −2.49 | 51.64 |
| | BLEND 11 at 16% | 81.55 | 16.96 | 67.78 | 69.87 | 75.95 | 9.14 | 19.05 | −3.07 | 33.85 |

All the 16% formulas listed in Table 25 are covering.

BLENDS 3 and 9 stand out in terms of the opacity imparted to the formulas which contain same. For the rest of the measurements and the observations with the naked eye, BLEND 3 is the closest to titanium dioxide. The BLENDS 9 and 11 have very slight differences in performance between them. The three BLENDS are good replacements for titanium dioxide, equivalent to 65%.

TABLE 26
Colorimetric measurements (average over three measurements) in a L*a*b*CIE 1976 chromatic space and
opacity of cosmetic formulations comprising yellow iron oxide as pigment and useful as lipstick

| | | | | | | | dE/ | dE/ | | |
|---|---|---|---|---|---|---|---|---|---|---|
| | | L*(D65) | a*(D65) | b*(D65) | C*(D65) | h(D65) | Bound+ | Bound− | dL | Opacity |
| Formula | TiO₂ LC981 at 16% (standard) | 79.54 | 7.88 | 31.38 | 32.35 | 75.91 | | / | / | 92.91 |
| at 16% | in the absence of whitening compound | 56.31 | 18.81 | 57.02 | 60.04 | 71.74 | −23.24 | 36.29 | / | 9.03 |
| | (negative bound) | | | | | | | | | |
| | BLEND 3 RAL at 16% | 72.39 | 12.38 | 41.97 | 43.76 | 73.56 | −7.16 | 13.55 | 22.94 | 58.68 |
| | BLEND 9 at 16% | 71.35 | 12.88 | 43.17 | 45.05 | 73.39 | −8.20 | 15.21 | 21.29 | 42.60 |
| | BLEND 11 at 16% | 71.55 | 12.77 | 42.84 | 44.70 | 73.40 | −7.99 | 14.80 | 21.68 | 65.21 |

All the 16% formulas listed in Table 26 are covering.

The three BLENDS are good alternatives to titanium dioxide, equivalent to 65% compared to a formula containing 16% titanium dioxide. BLEND 11 gives the best opacity to the formulation, but for the rest of the results, consistent with observations with the naked eye, BLEND 3 is closest to titanium dioxide.

TABLE 27
Colorimetric measurements (average over three measurements) in a L*a*b*CIE 1976 chromatic space and
opacity of cosmetic formulations comprising black iron oxide as pigment and useful as lipstick

| | | | | | | | dE/ | dE/ | | |
|---|---|---|---|---|---|---|---|---|---|---|
| | | L*(D65) | a*(D65) | b*(D65) | C*(D65) | h(D65) | Bound+ | Bound− | dL | Opacity |
| Formula | TiO₂ LC981 at 16% (standard) | 59.57 | −0.82 | −1.17 | 1.43 | 235.15 | | / | / | 98.67 |
| at 16% | in the absence of whitening compound | 20.69 | −0.76 | −3.99 | 4.06 | 259.27 | −38.88 | 38.99 | / | 13.20 |
| | (negative bound) | | | | | | | | | |
| | BLEND 3 RAL at 16% | 40.81 | −0.26 | −3.43 | 3.44 | 265.64 | −18.76 | 18.91 | 20.13 | 97.65 |
| | BLEND 9 at 16% | 39.69 | −0.26 | −3.33 | 3.34 | 265.57 | −19.88 | 20.00 | 19.02 | 93.61 |
| | BLEND 11 at 16% | 39.62 | −0.26 | −3.50 | 3.51 | 265.70 | −19.95 | 20.09 | 18.95 | 96.14 |

All the 16% formulas listed in Table 27 are covering.

The 3 BLENDS impart very good opacities to the formulas which contain same, the formula comprising BLEND 3 being even almost at the level of the formula comprising titanium dioxide. For the other results, there is little difference between BLEND 9 and BLEND 11. BLEND 3 gives the best performance.

In conclusion, of all the tests with formulas which can be used as lipstick, the three BLENDS 3, 9 and 11 are good replacements for titanium dioxide. The performance thereof varies depending on the pigment used in the formula, ranging from an equivalence of 50% to 80%.

### Example 3: Whitening Compositions for Solid Soap

The whitening compositions having the composition detailed in Table 28 were prepared.

TABLE 28
Compositions of the whitening compositions, the proportions
being expressed by weight/weight

| | BLEND | BLEND | BLEND | BLEND |
|---|---|---|---|---|
| Raw material | BNKAIN | BNMIIN | BNTCIN | BNIN |
| Boron nitride | 19 | 18 | 18 | 18 |
| Zinc oxide | 76 | 77 | 77 | 82 |
| Mica | — | 5 | — | — |
| Kaolin | 5 | — | — | — |
| Tricalcium citrate | — | — | 5 | — |
| ZnO/BN ratio | 4 | 4.28 | 4.28 | 4.56 |

### 3.1. Solid Soap Comprising 0.4% of Whitening Composition

### 3.1.1. Preparation of Solid Soaps

The different BLENDS were tested at a concentration of 0.4% by weight in the formula shown in detail in Table 29, in comparison with a formula comprising 0.4% by weight of anatase titanium dioxide (standard) and with a formula free of any whitening compound (negative bound). Cosmetic formulations having the compositions shown in detail in Table 29 were prepared.

Hereinafter, “soap base” corresponds to the soap base sold under the name Massa base 82/18 sem branqueador optico by Sinter Futura (INCI: Sodium Tallowate/Sodium Palm Kernelate/Sodium Chloride/Glycerin/Sodium Hydroxide/Tetrasodium EDTA/Etidronic Acid/Aqua.)

TABLE 29
Compositions of the cosmetic formulations useful as solid
soap, the proportions being expressed by weight/weight

| | | | Formula free |
|---|---|---|---|
| | | | of any whitening |
| | | 0.4% | compound |
| Phase | | formula | (negative bound) |
| A | Soap base | 94.60 | 95.00 |
| | Glycerin | 2.00 | 2.00 |
| | Paraffinum liquidum | 1.00 | 1.00 |
| | Propylene glycol | 2.00 | 2.00 |
| B | TiO₂ or one of the BLENDS | 0.40 | 0 |
| | from Table 28 | | |

### Procedure:

Weigh Phase A and mix same.
Add phase B to Phase A by passing the mixture 6 times in the laboratory extruder model
EJF46 from the company A2 Ingeniería Ltda, until complete homogenization.
Extrude the bar of solid soap.
Mold in a standard model press from the company A2 Ingeniería Ltda.

### 3.1.2. Colorimetric Measurements

Colorimetric measurements of the solid soaps were carried out with a Konica Minolta CM-3600d spectrophotometer.

Tables 30 and 31 below hereinbelow provide the colorimetric measurements measured on the cosmetic formulations listed in Table 29.

TABLE 30
Colorimetric measurements of the different formulas
in a L*a*b*CIE 1976 chromatic space.
The deltas are in comparison with the negative bound

| | Free of any | | | | |
|---|---|---|---|---|---|
| | whitening | | | | |
| | compound | | | | |
| | (negative | Unipure | Blend | Blend | Blend |
| | bound) | LC981 | BNKAIN | BNMIIN | BNTCIN |
| L* | 63.16 | 93.64 | 81.93 | 82.99 | 82.06 |
| a* | 1.78 | 2.20 | 1.18 | 1.07 | 0.87 |
| b | −19.48 | 9.19 | −13.41 | −12.25 | −12.45 |
| C* | 19.56 | 9.45 | 13.46 | 12.30 | 12.48 |
| delta | X | 30.48 | 18.77 | 19.83 | 18.90 |
| L* | | | | | |
| delta | X | 0.42 | −0.60 | −0.71 | −0.91 |
| a* | | | | | |
| delta | X | 28.67 | 6.07 | 7.23 | 7.03 |
| b* | | | | | |
| delta | X | −10.11 | −6.1 | −7.26 | −7.08 |
| C* | | | | | |
| delta | X | 929.03 | 352.31 | 393.23 | 357.21 |
| L\hat{ }2 | | | | | |
| delta | X | 0.18 | 0.36 | 0.50 | 0.83 |
| a\hat{ }2 | | | | | |
| delta | X | 821.97 | 36.85 | 52.28 | 49.42 |
| b\hat{ }2 | | | | | |
| sum | X | 1751.18 | 389.52 | 446.01 | 407.46 |
| delta | X | 41.85 | 19.74 | 21.12 | 20.19 |
| E* | | | | | |

TABLE 31
Colorimetric measurements of the different formulas in a
L*a*b*CIE 1976 chromatic space. The deltas are in comparison
with the formulation comprising titanium dioxide.

| | | Free of any | | | |
|---|---|---|---|---|---|
| | Unipure | whitening compound | Blend | Blend | Blend |
| | LC981 | (negative bound) | BNKAIN | BNMIIN | BNTCIN |
| L* | 93.64 | 63.16 | 81.93 | 82.99 | 82.06 |
| a* | 2.2 | 1.78 | 1.18 | 1.07 | 0.87 |
| b | 9.19 | −19.48 | −13.41 | −12.25 | −12.45 |
| C* | 9.45 | 19.56 | 13.46 | 12.30 | 12.48 |
| delta | X | −30.48 | −11.71 | −10.65 | −11.58 |
| L* | | | | | |
| delta | X | −0.42 | −1.02 | −1.13 | −1.33 |
| a* | | | | | |
| delta | X | −28.67 | −22.6 | −21.44 | −21.64 |
| b* | | | | | |
| delta | X | 10.11 | 4.01 | 2.85 | 3.03 |
| C* | | | | | |
| delta | X | 929.03 | 137.12 | 113.42 | 134.10 |
| L\hat{ }2 | | | | | |
| delta | X | 0.18 | 1.04 | 1.28 | 1.77 |
| a\hat{ }2 | | | | | |
| delta | X | 821.97 | 510.76 | 459.67 | 468.29 |
| b\hat{ }2 | | | | | |
| sum | X | 1751.18 | 648.92 | 574.37 | 604.15 |
| delta | X | 41.85 | 25.47 | 23.97 | 24.58 |

The parameter L is the parameter used for evaluating the whitening character for solid soaps. The 3 BLENDS, used at 0.4% in the solid soap formulation provide very good lightness. The blends can be considered as alternatives to titanium dioxide, with an equivalence of 50%.

### 3.1.3. Mushing

Principle of the mushing test: a thick, soft substance formed on the surface of the soap and which affects the longevity of the bar.

### Procedure:

Put the bar of soap in a beaker and add 200 ml of water. Leave the soap bar immersed for 24 hours. Remove the soap bar and let same drain for 30 min. Leave the beaker at 110° C. in the oven for 24 hours. Weigh the dried beaker. The result is expressed as a percentage.

TABLE 32
Mushing of solid soaps

| Soap bar | % Mushing |
|---|---|
| Free of any whitening compound (negative bound) | 7.53 |
| BNTCIN | 12.06 |
| BNMIIN | 11.83 |
| BNKAIN | 11.54 |
| UNIPURE WHITE LC981 | 10.72 |

Although slightly higher than the results with titanium dioxide, the mushing of soap bars from formulas with BLENDS remains within the normal range of a soap bar.

### 3.1.4. Hardness/Cracking Test

Principle of the hardness/cracking test: occurrence of cracks in the soap bar due to moisture loss

### Procedure:

Immerse the bar of soap in 200 ml of water
After 24 hours, take out the bar of soap and dry same at 60° C.
Evaluate the number of cracks present on the bar of soap at 24 h and 72 h using a crack scale from 1 to 5, 5 being the most cracked and 1 the least cracked.
Coloring the soap bar with a Blue 1 coloring agent makes it easier to read cracks.

TABLE 33
Crack test results

| | Crack scale | |
|---|---|---|
| Soap bar: | 24 hrs | 72 hrs |
| Free of any whitening compound (negative bound) | 1 | 2 |
| Comprising BNTCIN | 1 | 2 |
| Comprising BNMIIN | 1 | 2 |
| Comprising BNKAIN | 1 | 2 |
| Comprising UNIPURE WHITE LC981 (standard) | 2 | 3 |

Formulas with BLENDS have fewer cracks than formula with titanium dioxide.

### 3.1.5. Foam Test

Principle of the foam test: foam formed on the surface of the soap

### Procedure:

Weigh 0.5 g of soap and dissolve same in 200 ml of water.
Take 50 ml of solution and place same in a measuring cylinder.
Shake the cylinder 10 times and measure the volume of the foam

TABLE 34
Foam test results

| Soap bar: | Foam volume (ml) |
|---|---|
| Free of any whitening compound (negative bound) | 45 |
| Comprising BNTCIN | 43 |
| Comprising BNMIIN | 40 |
| Comprising BNKAIN | 39 |
| Comprising UNIPURE WHITE LC981 (standard) | 41 |

The BLENDS have no influence on the volume of foam.

In conclusion, the 3 BLENDS BNTCIN, BNMIIN, BNKAIN are good alternatives to titanium dioxide. Introduced at 0.4% into the formulation useful as a solid soap, there is a 50% equivalence compared to a solid soap containing TiO₂ and same have no influence on the final properties of the soap bar.

3.2. Solid Soaps with Different Formulas

The following tests were carried out in order to verify whether the results obtained depend on the composition of the solid soap formulation.

Solid soaps comprising different percentages of whitening composition were prepared. The different BLENDS were tested at different percentages by weight in the formulae given in detail in Table 28, in comparison with a formula comprising 0.4% by weight of anatase titanium dioxide (standard) and with a formula free of any whitening compound (negative bound). Cosmetic formulations having the compositions shown in detail in Table 35 were prepared.

TABLE 35
Composition of solid soaps

| | | | | | | | | Formula |
|---|---|---|---|---|---|---|---|---|
| | | | | | | | | free of |
| | | | | | | | | any |
| | | | | | | | | whitening |
| | | | | | | | Formula | compound |
| | 0.2% | 0.4% | 0.6% | 0.8% | 1% | 1.5% | with | (negative |
| | formula | formula | formula | formula | formula | formula | TiO₂ | bound) |
| Soap base* | 99.8 | 99.6 | 99.4 | 99.2 | 99 | 98.5 | 99.6 | 100 |
| One of the | 0.2 | 0.4 | 0.6 | 0.8 | 1 | 1.5 | | |
| BLENDS from | | | | | | | | |
| Table 34 | | | | | | | | |
| Unipure | | | | | | | 0.4 | |
| White | | | | | | | | |
| LC981(Titanium | | | | | | | | |
| dioxide) | | | | | | | | |

*soap base Viruta de jabon from Fabrica de Jabon La Corona

### Procedure:

Mix and extrude soap bars with the laboratory extruder model EJF46 from the company A2 Ingeniería Ltda.

Mold in a standard model press from the company A2 Ingeniería Ltda.

Table 36 gives the lightness values L* of the solid soaps measured with a Konica Minolta CM-3600d spectrophotometer.

TABLE 36
Lightness measures L* of the different solid soap formulas.

| | Free of any | | | | | |
|---|---|---|---|---|---|---|
| | whitening | | | | | |
| | compound | | | | | |
| | (negative | Unipure | Blend | Blend | Blend | Blend |
| % | bound) | LC981 | BNIN | BNKAIN | BNMIIN | BNTCIN |
| 0 | 84.4 | — | — | — | — | — |
| 0.2 | — | — | 85.63 | 85.96 | 86.77 | 86.28 |
| 0.4 | — | 91.54 | 87.39 | 87.87 | 87.42 | 87.24 |
| 0.6 | — | — | 88.13 | 88.18 | 87.64 | 87.55 |
| 0.8 | — | — | 89.15 | 88.85 | 88.16 | 88.53 |
| 1 | — | — | 89.77 | 90.37 | 89.56 | 89.33 |
| 1.5 | — | — | 91.61 | 91.07 | 90.74 | 88.17 |

The 4 BLENDS impart a very good lightness to the solid soap formulation. Same can all be considered as alternatives to titanium dioxide. BNIN and BNKAIN BLENDS giving the best performance and being the most consistent from 0.4% on and achieving almost the same result as titanium dioxide at 1.5%.

### Example 4: Whitening Compositions for Compact Powders

### 4.1. Preparation of Whitening Compositions

The whitening compositions having the composition detailed in Table 37 were prepared.

TABLE 37
Compositions of the whitening compositions, the proportions
being expressed by weight/weight

| | BLEND 4 PP | BLEND 4K PP | BLEND 9 PP NEW |
|---|---|---|---|
| Boron nitride BN02 | 36 | 36 | 36 |
| Zinc oxide | 64 | 34 | 59 |
| Kaolin | 0 | 30 | 5 |
| ZnO/BN ratio | 1.78 | 0.94 | 1.64 |

### 4.2. Preparation of Cosmetic Formulations Useful as Compact Powders

The compositions of the cosmetic formulations which are useful as prepared compact powders are given in detail in Table 38.

TABLE 38
Compositions of the cosmetic formulations useful as compact powders

| | | | | 20% |
|---|---|---|---|---|
| Phase | Brand name | INCI | Supplier | formula |
| A | Unipure Yellow LC182 | CI77492 | Sensient | 3.6 |
| | Unipure Red LC381 | CI 77491 | Sensient | 1.30 |
| | Unipure Black LC989 | CI 77499 | Sensient | 0.40 |
| | Mica 8 | Mica | Sensient | 25.00 |
| | Magnesium stearate | Magnesium stearate | Univar | 10.00 |
| B | LCW talcum | Talcum | Sensient | 37.70 |
| | Unipure White LC981, | CI 77891 (titanium dioxide), boron | Sensient | 20.00 |
| | or Caress BN02, or | nitride, zinc oxide, one of the BLENDs | | |
| | zinc oxide, or one of | listed in Table 37 | | |
| | the BLENDs listed in | | | |
| | table 37 | | | |
| C | Jeechem OSS | Octyldodecyl stearoyl stearate | Jeen | 2.00 |
| | | | International | |

### Procedure:

Mix the phase A for 30 seconds, 3 times.
Add phase B, mix for 30 seconds, 2 times
Add phase C, mix for 30 seconds, 3 times
Sieve
Compact at 200 psi for 1 sec with a Cavalla compactor

### 4.3. Colorimetric Results

The colorimetric and coverage parameters are of equal significance in such application.

Table 39 below gives the colorimetric measurements measured with a Konica Minolta CM-5 spectrophotometer on the cosmetic formulations listed in Table 38.

TABLE 39
Colorimetric and coverage measurements (average over three measurements)
of the different formulas in a L*a*b*CIE 1976 chromatic space.

| | | L* | a* | b* | C* | H | dE* | Coverage |
|---|---|---|---|---|---|---|---|---|
| Formula comprising: | Unipure White | 73.26 | 11.3 | 18.54 | 21.71 | 58.63 | / | covering |
| | LC981 | | | | | | | |
| | (standard) | | | | | | | |
| | Boron nitride alone | 72.23 | 11.81 | 19.25 | 22.58 | 58.47 | 1.35 | covering |
| | (comparative) | | | | | | | |
| | Zinc oxide alone | 69.94 | 12.16 | 19.31 | 22.82 | 57.79 | 3.51 | moderately |
| | (comparative) | | | | | | | covering |
| | Kaolin (comparative) | 70.15 | 12.8 | 21.51 | 21.71 | 25.03 | 3.2 | moderately |
| | | | | | | | | covering |
| | Blend 4 PP | 70.86 | 12.39 | 19.95 | 23.48 | 58.16 | 2.98 | covering |
| | Blend 4 K PP | 70.68 | 12.54 | 20.71 | 24.21 | 58.8 | 3.6 | covering |
| | Blend 9 PP NEW | 70.31 | 12.42 | 20.45 | 24 | 58.75 | 3.2 | covering |

The formulation comprising boron nitride alone has results very close to titanium dioxide but, at the sensory level, there is no equivalence. When applied, the formulation comprising zinc oxide alone is too dark and transparent. The formulation comprising kaolin alone is too yellow.

Formulations comprising BLEND 4PP, BLEND 4K PP and BLEND 9 PP NEW are good alternatives to titanium dioxides with an equivalence between 85 and 90% compared to a formula comprising 20% of titanium dioxide. The formulations giving the best results in terms of lightness and coverage are the formulations comprising blend 4PP and blend 4K PP.

### Example 5: Comparison of the Opacities of the Whitening Compositions with the Opacities of the Raw Materials Used Alone

Opacity tests were carried out on the whitening compositions with a composition given in detail in the preceding examples and, as a comparison, on the raw materials used alone.

Method for evaluating opacity in a 40% oily medium:

Grind (3×50 rotations), in an Engelsmann JEL25/53 pigment mill, a mixture of 40% of one of the BLENDS or boron nitride, zinc oxide, kaolin or tricalcium citrate with 60% linseed oil.

Method for evaluating opacity in a 20% aqueous media:

1% sodium polyacrylate (Covacryl RH) is put into the water to form a gel. 80% of the gel is mixed with 20% of one of the BLENDS or boron nitride, zinc oxide, kaolin or tricalcium citrate.

Using a film-stretcher, a 76.2-micron thick film was stretched over a black and white contrast card (Black & White Leneta Contrast Card) and the opacity was measured according to the method described hereinabove.

The results are shown in Table 40. A result lower than 50 corresponds to a transparent film and a result higher than 50 corresponds to an opaque film.

TABLE 40
Opacity of whitening compositions or of raw materials.

| | | Opacity | Opacity in a 20% |
|---|---|---|---|
| | | in a 40% oily | aqueous |
| Identity | Supplier Name | medium | medium |
| Foundation | BLEND 4 | 82.81 | 76.20 |
| | BLEND 9 NEW | 81.49 | 79.98 |
| | BLEND 8 NEW | 79.53 | 76.32 |
| Lipstick | BLEND 3 | 82.18 | 79.30 |
| | BLEND 9 | 79.23 | 77.57 |
| | BLEND 11 | 77.97 | 74.15 |
| Solid soaps | BLEND BNKAIN | 79.9 | 75.24 |
| | BLEND BNMIIN | 79.8 | 75.72 |
| | BLEND BNTCIN | 79.16 | 73.2 |
| | BLEND BNIN | 79.64 | 78.73 |
| Raw materials | Boron nitride | 80.68 | 65.74 |
| | Zinc oxide | 69.01 | 61.91 |
| | Kaolin | 11.85 | 22.09 |
| | Tricalcium citrate | 4.48 | 16.84 |
| | Kaolin | 11.85 | 22.09 |
| | Tricalcium citrate | 4.48 | 16.84 |

Table 41 below makes it possible to compare the theoretical opacities (obtained by calculation from the opacities of the raw materials taking into account the respective proportions thereof) and the observed opacities.

TABLE 41
Comparison of theoretical opacities (obtained by calculation from the opacities of the raw
materials taking into account the respective proportions thereof) and observed opacities

| | | BLEND | | | | | | | | | | | |
|---|---|---|---|---|---|---|---|---|---|---|---|---|---|
| | | | 8 | 9 | | | | | | | | nitrure de | oxyde de |
| | | 4 | NEW | NEW | 3 | 9 | 11 | | | | BLEND | boron nitride | zinc oxide |
| | | FDT | FDT | FDT | RAL | RAL | RAL | BNKAIN | BNMIIN | BNTCIN | BNIN | (ref.) | (ref) |
| Boron nitride (%) | | 36 | 26 | 36 | 27 | 28 | 26 | 19 | 18 | 18 | 18 | 100 | 0 |
| ZnO (%) | | 64 | 69 | 59 | 73 | 52 | 54 | 76 | 77 | 77 | 82 | 0 | 100 |
| Opacity | Theoretical* | 73.2 | 68.6 | 69.8 | 72.2 | 58.5 | 58.2 | 67.8 | 67.7 | 67.7 | 71.1 | 80.68 | 69.01 |
| in a | observed | 82.81 | 79.53 | 81.49 | 82.18 | 79.23 | 77.97 | 79.9 | 79.8 | 79.16 | 79.64 | 80.68 | 69.01 |
| 40% | | | | | | | | | | | | | |
| oily | | | | | | | | | | | | | |
| medium | | | | | | | | | | | | | |
| Opacity | Theoretical** | 63.6 | 59.8 | 60.1 | 62.9 | 50.6 | 50.5 | 59.5 | 59.5 | 59.5 | 62.6 | 65.74 | 61.91 |
| in a | observed | 76.2 | 76.32 | 79.98 | 79.3 | 77.57 | 74.15 | 75.24 | 75.72 | 73.2 | 78.73 | 65.74 | 61.91 |
| 20% | | | | | | | | | | | | | |
| aqueous | | | | | | | | | | | | | |
| medium | | | | | | | | | | | | | |

*Theoretical opacity calculated by [(boron nitride concentration in the BLEND)*80.68 + (ZnO concentration in the BLEND)*69.01]/100
**Theoretical opacity calculated by [(boron nitride concentration in the BLEND) *65.74 + (ZnO concentration in the BLEND)*61.91]/100

The results of Table 41 show that, in all cases, the observed opacity is greater than the theoretical opacity, i.e. the expected opacity by cumulating the opacities of each of the raw materials ZnO and boron nitride, taking into account the respective proportions thereof in each BLEND. The above shows a synergistic effect of the combined use of boron nitride and of zinc oxide, on the imparted opacity.

1. A method for preparing a cosmetic formulation, comprising adding to the cosmetic formulation a whitening composition, said whitening composition comprising:
from 0 to 30% by weight of a whitening agent selected from kaolin optionally calcined, cerium oxide, a citrate salt, mica and a mixture thereof,
from 70 to 100% by weight of a mixture of zinc oxide and of boron nitride, the proportions being with respect to the cumulative weight of whitening agent, of zinc oxide and of boron nitride,
wherein the ratio of the weight of zinc oxide to the weight of boron nitride is 0.9 to 5.0, the whitening composition being free of titanium dioxide.

2. The method according to claim 1, wherein, in the whitening composition:
the boron nitride is in the form of particles with a mean diameter, as measured by laser diffraction, from 1 to 30 μm, and/or
the zinc oxide is in the form of particles with a mean diameter, as measured by laser diffraction, from 30 nm to 20 μm.

3. The method according to claim 1, wherein the ratio between the weight of zinc oxide and the weight of boron nitride of the whitening composition is from 0.9 to 3.0.

4. The method according to claim 1, wherein the whitening composition comprises:
from 0 to 10% by weight of a whitening agent selected from kaolin optionally calcined, cerium oxide and a mixture thereof,
from 90 to 100% by weight of a mixture of zinc oxide and of boron nitride,
the proportions being with respect to the cumulative weight of whitening agent, of zinc oxide and of boron nitride,
wherein the ratio between the weight of zinc oxide and the weight of boron nitride is from 0.9 to 3.0.

5. The method according to claim 1, wherein the whitening composition comprises:
from 0 to 25% by weight of a whitening agent chosen from kaolin optionally calcined, a citrate salt and a mixture thereof,
from 75 to 100% by weight of a mixture of zinc oxide and of boron nitride, the proportions being with respect to the cumulative weight of whitening agent, of zinc oxide and of boron nitride,
wherein the ratio of the weight of zinc oxide to the weight of boron nitride is from 1.5 to 3.0.

6. The method according to claim 1, wherein the whitening composition comprises:
from 0 to 10% by weight of a whitening agent chosen from kaolin optionally calcined, a citrate salt, mica and a mixture thereof,
from 90 to 100% by weight of a mixture of zinc oxide and of boron nitride, the proportions being with respect to the cumulative weight of whitening agent, of zinc oxide and of boron nitride,
wherein the ratio of the weight of zinc oxide to the weight of boron nitride is from 3.5 to 5.0.

7. The method according to claim 1, wherein the whitening composition comprises:
from 0 to 30% by weight of a whitening agent chosen from kaolin optionally calcined, cerium oxide and a mixture thereof,
from 70 to 100% by weight of a mixture of zinc oxide and of boron nitride,
the proportions being with respect to the cumulative weight of whitening agent, of zinc oxide and of boron nitride,
wherein the ratio between the weight of zinc oxide and the weight of boron nitride is from 0.9 to 3.0.

8. The method according to claim 7, wherein the whitening composition comprises:
from 1 to 30% by weight of a whitening agent chosen from kaolin optionally calcined, cerium oxide and a mixture thereof,
from 70 to 99% by weight of a mixture of zinc oxide and of boron nitride, the proportions being with respect to the cumulative weight of whitening agent, of zinc oxide and of boron nitride,
wherein the ratio of the weight of zinc oxide to the weight of boron nitride is from 0.9 to 2.0.

9. The method according to claim 7, wherein the whitening composition comprises:
from 0 to 10% by weight of a whitening agent chosen from kaolin optionally calcined, cerium oxide and a mixture thereof,
from 32 to 40% by weight of boron nitride, and
from 55 to 63% by weight of zinc oxide,
the proportions being with respect to the cumulative weight of whitening agent, of zinc oxide and of boron nitride,
wherein the ratio of the weight of zinc oxide to the weight of boron nitride is from 0.9 to 2.0.

10. The method according to claim 1, for the preparation of a cosmetic formulation which is a compact powder, a lipstick or a foundation.

11. A whitening composition comprising:
from 1 to 30% by weight of a whitening agent chosen from kaolin optionally calcined, cerium oxide and a mixture thereof,
from 70 to 99% by weight of a mixture of zinc oxide and of boron nitride, the proportions being with respect to the cumulative weight of whitening agent, of zinc oxide and of boron nitride,
wherein the ratio of the weight of zinc oxide to the weight of boron nitride is from 0.9 to 3.0, the whitening composition being free of titanium dioxide.

12. The whitening composition according to claim 11, wherein the ratio between the weight of zinc oxide and the weight of boron nitride of the whitening composition is from 0.9 to 2.0.

13. A cosmetic formulation comprising a whitening composition according to claim 11, the cosmetic formulation being free of titanium dioxide.

14. The cosmetic formulation according to claim 13, which is a compact powder, and which comprises from 10 to 30% by weight of the whitening composition with respect to the weight of the cosmetic formulation.

15. The method according to claim 4, wherein the ratio between the weight of zinc oxide and the weight of boron nitride is from 1.3 to 3.0.

16. The method according to claim 4, wherein, in the whitening composition:
the boron nitride is in the form of particles with a mean diameter, as measured by laser diffraction, from 1 to 30 μm, and/or
the zinc oxide is in the form of particles with a mean diameter, as measured by laser diffraction, from 30 nm to 20 μm.

17. The method according to claim 5, wherein, in the whitening composition:
the boron nitride is in the form of particles with a mean diameter, as measured by laser diffraction, from 1 to 30 μm, and/or
the zinc oxide is in the form of particles with a mean diameter, as measured by laser diffraction, from 30 nm to 20 μm.

18. The method according to claim 6, wherein, in the whitening composition:
the boron nitride is in the form of particles with a mean diameter, as measured by laser diffraction, from 1 to 30 μm, and/or
the zinc oxide is in the form of particles with a mean diameter, as measured by laser diffraction, from 30 nm to 20 μm.

19. The method according to claim 7, wherein the ratio between the weight of zinc oxide and the weight of boron nitride is from 0.9 to 2.0.

20. The method according to claim 7, wherein, in the whitening composition:
the boron nitride is in the form of particles with a mean diameter, as measured by laser diffraction, from 1 to 30 μm, and/or
the zinc oxide is in the form of particles with a mean diameter, as measured by laser diffraction, from 30 nm to 20 μm.
